SEQUENCE LISTING (129 sequences)

SEQ ID NO: 1 - protein, 584 aa, synthetic construct
METDTLLLWVLLLWVPGSTGDSLSDKKSLLLPPTNFTIKVTGLSQVLLRWESNPDQEPVNIELGYHVRINAPQEEDYETRNTESKRVTVLHKGFSASVQTVPWNGQPLPPSRWVSAELKAPPGSPGTSIVNLTCTTNTTVNNHTPLRPYQVSLHCSWLAGKDAPEDTQYFLYYRYGSWTEECQEYRRDTLNRNTACWFPKTFISSRGSEALAVHANGSSKHAPIKPFDQLFALHAIDRINPPVNVTAEIEGTCLSIQWEKPVSAFPIHCFEYEVKIYNTRKKYFQTEKLMTNAFISIIDDISKYSVQVRATVSSMCRQMGFWSEWSQPIYVGGKPIPNPLLGLDSTENLYFQGEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 2 - protein, 585 aa, synthetic construct
MGWSCIILFLVATATGVHSDSLSDKKSLLLPPTNFTIKVTGLSQVLLRWESNPDQEPVNIELGYHVRINAPQEEDYETRNTESKRVTVLHKGFSASVQTVPWNGQPLPPSRWVSAELKAPPGSPGTSIVNLTCTTNTTVNNHTPLRPYQVSLHCSWLAGKDAPEDTQYFLYYRYGSWTEECQEYRRDTLNRNTACWFPKTFISSRGSEALAVHANGSSKHAPIKPFDQLFALHAIDRINPPVNVTAEIEGTCLSIQWEKPVSAFPIHCFEYEVKIYNTRKKYFQTEKLMTNAFISIIDDISKYSVQVRATVSSMCRQMGFWSEWSQPIYVGGGGSGGGSEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGGLNDIFEAQKIEWHE

SEQ ID NO: 3 - protein, 119 aa, synthetic construct
EVQLVESGGDLVKPGGSLKLSCAASGFTFSNYAMSWVRQTPDKRLEWVATIGSGGHYTYHPDSVKGRFSISRDNAKNTLSLQMSSLKSEDTAMYYCTRETDGYDGMDYWGQGTSVTVSS

SEQ ID NO: 4 - protein, 107 aa, synthetic construct
DIVMTQSPATLSVTPGDRVSLSCRASQSISDYLYWYQQKSHESPRLLIKYASQSISGIPARFSGSGSGSDFTLSINSVEPEDVGVYYCQNGHSFPYTFGGGTKLEIK

SEQ ID NO: 5 - protein, 119 aa, synthetic construct
DVQLVESGGDLVKPGGSLRLTCVGSGFTFSNYAMSWVRQAPGEGLQWVATIGSGGHYTYHPDSVKGRFTISRDNAKNTLYLQMNSLQTEDTASYYCTRETDGYDGMDYWGQGTLVTVSS

SEQ ID NO: 6 - protein, 107 aa, synthetic construct
EIQMTQSPSSLSASVGDRVTITCRASQSISDYLYWYQQKPGKAPKLLIYYASQSISGVPSRFSGSGSGTDFTLTISSLQPEDFATYYCQNGHSFPYTFGPGTKLEIK

SEQ ID NO: 7 - protein, 107 aa, synthetic construct
DIVMTQTPLSLSVTPGEPASISCRASQSISDYLYWYLQKPGQSPRRLIYYASQSISGVPDRFSGSGSGTDFTLRISRVEADDVGVYYCQNGHSFPYTFGPGTKLEIK

SEQ ID NO: 8 - protein, 119 aa, synthetic construct
DVQLVESGGDLVKPGGSLRLTCVGSGFTFSNYAMSWVRQAPGEGLQWVATIGSGGHYTYHPDSVKGRFTISRDNAKNTLYLQMNSLQTEDTASYYCTRETDGYDGLDVWGQGTLVTVSS

SEQ ID NO: 9 - protein, 107 aa, synthetic construct
DIVMTQTPLSLSVTPGEPASISCRASQSISDYLYWYLQKPGQSPRRLIYYASQSISGVPDRFSGSGSGTDFTLRISRVEADDVGVYYCQQGHSFPYTFGPGTKLEIK

SEQ ID NO: 10 - protein, 107 aa, synthetic construct
DIVMTQTPLSLSVTPGEPASISCRASQSISDYLYWYLQKPGQSPRRLIYYASQSISGVPDRFSGSGSGTDFTLRISRVEADDVGVYYCQSGHSFPYTFGPGTKLEIK

SEQ ID NO: 11 - protein, 119 aa, synthetic construct
DVQLVESGGDLVKPGGSLRLTCVGSGFTFSNYAMSWVRQAPGEGLQWVATIGSGGHYTYHPDSVKGRFTISRDNAKNTLYLQMNSLQTEDTASYYCTRETDGYGPMDVWGQGTLVTVSS

SEQ ID NO: 12 - protein, 107 aa, synthetic construct
DIVMTQTPLSLSVTPGEPASISCRASQSISDYLYWYLQKPGQSPRRVFYTDSAEVSGVPDRFSGSGSGTDFTLRISRVEADDVGVYYCQNGHSFPYTFGPGTKLEIK

SEQ ID NO: 13 - protein, 107 aa, synthetic construct
DIVMTQTPLSLSVTPGEPASISCRASQSISDYLYWYLQKPGQSPRRIFFTESQRASGVPDRFSGSGSGTDFTLRISRVEADDVGVYYCQNGHSFPYTFGPGTKLEIK

SEQ ID NO: 14 - protein, 107 aa, synthetic construct
DIVMTQTPLSLSVTPGEPASISCRASQSISDYLYWYLQKPGQSPRRIFFASSLYDSGVPDRFSGSGSGTDFTLRISRVEADDVGVYYCQNGHSFPYTFGPGTKLEIK

SEQ ID NO: 15 - protein, 107 aa, synthetic construct
DIVMTQTPLSLSVTPGEPASISCRASQSISDYLYWYLQKPGQSPRRIFFGYSGTKSGVPDRFSGSGSGTDFTLRISRVEADDVGVYYCQNGHSFPYTFGPGTKLEIK

SEQ ID NO: 16 - protein, 107 aa, synthetic construct
DIVMTQTPLSLSVTPGEPASISCRASQSISDYLYWYLQKPGQSPRRVFFTVSGGVSGVPDRFSGSGSGTDFTLRISRVEADDVGVYYCQNGHSFPYTFGPGTKLEIK

SEQ ID NO: 17 - protein, 107 aa, synthetic construct
DIVMTQTPLSLSVTPGEPASISCRASQSISDYLYWYLQKPGQSPRRVFFTPSDSASGVPDRFSGSGSGTDFTLRISRVEADDVGVYYCQNGHSFPYTFGPGTKLEIK

SEQ ID NO: 18 - protein, 107 aa, synthetic construct; identical to SEQ ID NO: 17

SEQ ID NO: 19 - protein, 119 aa, synthetic construct; identical to SEQ ID NO: 11

SEQ ID NO: 20 - protein, 119 aa, synthetic construct
DVQLVESGGDLVKPGGSLRLTCVGSGFTFSNYAMSWVRQAPGEGLQWVATIGSGGHYTYHPDSVKGRFTISRDNAKNTLYLQMNSLQTEDTASYYCTRETDGYGPLDVWGQGTLVTVSS

SEQ ID NO: 21 - protein, 107 aa, synthetic construct
EIQMTQSPSSLSASVGDRVTITCRASQSISDYLYWYQQKPGKAPKLVFYTDSAEVSGVPSRFSGSGSGTDFTLTISSLQPEDFATYYCQQGHSFPYTFGPGTKLEIK

SEQ ID NO: 22 - protein, 107 aa, synthetic construct
EIQMTQSPSSLSASVGDRVTITCRASQSISDYLYWYQQKPGKAPKLVFYTDSAEVSGVPSRFSGSGSGTDFTLTISSLQPEDFATYYCQSGHSFPYTFGPGTKLEIK

SEQ ID NO: 23 - protein, 107 aa, synthetic construct
EIQMTQSPSSLSASVGDRVTITCRASQSISDYLYWYQQKPGKAPKLLIYYASQSISGVPSRFSGSGSGTDFTLTISSLQPEDFATYYCQQGHSFPYTFGPGTKLEIK

SEQ ID NO: 24 - protein, 119 aa, synthetic construct
DVQLVESGGDLVKPGGSLRLTCVGSGFTFSNYAMSWVRQAPGEGLQWVATIGSGGHYTYHPDSVKGRFTISRDNAKNTLYLQMNSLQTEDTASYYCTRETDGYGPILDWGQGTLVTVSS

SEQ ID NO: 25 - protein, 107 aa, synthetic construct
DIVMTQTPLSLSVTPGEPASISCRASQSISDYLYWYLQKPGQSPRLLFNVLSQRSSGVPDRFSGSGSGTDFTLRISRVEADDVGVYYCQQGLVFPYTFGPGTKLEIK

SEQ ID NO: 26 - protein, 119 aa, synthetic construct
DVQLVESGGDLVKPGGSLRLTCVGSGFTFSNYAMSWVRQAPGEGLQWVATIGSGGHYTYHPDSVKGRFTISRDNAKNTLYLQMNSLQTEDTASYYCTRETDGYGPLDLWGQGTLVTVSS

SEQ ID NO: 27 - protein, 107 aa, synthetic construct
DIVMTQTPLSLSVTPGEPASISCRASQSISDYLYWYLQKPGQSPRLLFNSVSPTRSGVPDRFSGSGSGTDFTLRISRVEADDVGVYYCQQGVVFPYTFGPGTKLEIK

SEQ ID NO: 28 - protein, 119 aa, synthetic construct; identical to SEQ ID NO: 24

SEQ ID NO: 29 - protein, 107 aa, synthetic construct
DIVMTQTPLSLSVTPGEPASISCRASQSISDYLYWYLQKPGQSPRLIINYGSVLLSGVPDRFSGSGSGTDFTLRISRVEADDVGVYYCQQGLLFPYTFGPGTKLEIK

SEQ ID NO: 30 - protein, 119 aa, synthetic construct; identical to SEQ ID NO: 26

SEQ ID NO: 31 - protein, 107 aa, synthetic construct
DIVMTQTPLSLSVTPGEPASISCRASQSISDYLYWYLQKPGQSPRLIINPMSGQLSGVPDRFSGSGSGTDFTLRISRVEADDVGVYYCQQGLLFPYTFGPGTKLEIK

SEQ ID NO: 32 - protein, 119 aa, synthetic construct; identical to SEQ ID NO: 24

SEQ ID NO: 33 - protein, 107 aa, synthetic construct
DIVMTQTPLSLSVTPGEPASISCRASQSISDYLYWYLQKPGQSPRRLIYFGSVRSSGVPDRFSGSGSGTDFTLRISRVEADDVGVYYCQQGLQFPYTFGPGTKLEIK

SEQ ID NO: 34 - skipped

SEQ ID NO: 35 - protein, 119 aa, synthetic construct; identical to SEQ ID NO: 24

SEQ ID NO: 36 - protein, 119 aa, synthetic construct; identical to SEQ ID NO: 24

SEQ ID NO: 37 - protein, 107 aa, synthetic construct
DIVMTQTPLSLSVTPGEPASISCRASQSISDYLYWYLQKPGQSPRRIIFTGSQRNSGVPDRFSGSGSGTDFTLRISRVEADDVGVYYCQQGAFFPYTFGPGTKLEIK

SEQ ID NO: 38 - protein, 119 aa, synthetic construct; identical to SEQ ID NO: 24

SEQ ID NO: 39 - protein, 107 aa, synthetic construct
DIVMTQTPLSLSVTPGEPASISCRASQSISDYLYWYLQKPGQSPRRIFNHLSPSSSGVPDRFSGSGSGTDFTLRISRVEADDVGVYYCQQGSVFPYTFGPGTKLEIK

SEQ ID NO: 40 - protein, 119 aa, synthetic construct; identical to SEQ ID NO: 24

SEQ ID NO: 41 - protein, 107 aa, synthetic construct
DIVMTQTPLSLSVTPGEPASISCRASQSISDYLYWYLQKPGQSPRRVFFHSSYRQSGVPDRFSGSGSGTDFTLRISRVEADDVGVYYCQQGMLFPFTFGPGTKLEIK

SEQ ID NO: 42 - protein, 119 aa, synthetic construct; identical to SEQ ID NO: 24

SEQ ID NO: 43 - protein, 107 aa, synthetic construct
DIVMTQTPLSLSVTPGEPASISCRASQSISDYLYWYLQKPGQSPRLIINQASSKLSGVPDRFSGSGSGTDFTLRISRVEADDVGVYYCQQGPLFPYTFGPGTKLEIK

SEQ ID NO: 44 - protein, 119 aa, synthetic construct
DVQLVESGGDLVKPGGSLRLTCVGSGFTFSNYAMSWVRQAPGEGLQWVATIGSGGHYTYHPDSVKGRFTISRDNAKNTLYLQMNSLQTEDTASYYCTRETDGYGPMLDWGQGTLVTVSS

SEQ ID NO: 45 - protein, 107 aa, synthetic construct
DIVMTQTPLSLSVTPGEPASISCRASQSISDYLYWYLQKPGQSPRLLFNYGSVLASGVPDRFSGSGSGTDFTLRISRVEADDVGVYYCQQGYLFPYTFGPGTKLEIK

SEQ ID NO: 46 - protein, 119 aa, synthetic construct; identical to SEQ ID NO: 26

SEQ ID NO: 47 - protein, 107 aa, synthetic construct
DIVMTQTPLSLSVTPGEPASISCRASQSISDYLYWYLQKPGQSPRRIIFTGSQRNSGVPDRFSGSGSGTDFTLRISRVEADDVGVYYCQQGMVFPYTFGPGTKLEIK

SEQ ID NO: 48 - protein, 119 aa, synthetic construct; identical to SEQ ID NO: 24

SEQ ID NO: 49 - protein, 107 aa, synthetic construct
DIVMTQTPLSLSVTPGEPASISCRASQSISDYLYWYLQKPGQSPRRVIYTGSQRSSGVPDRFSGSGSGTDFTLRISRVEADDVGVYYCQQGLVFPYTFGPGTKLEIK

SEQ ID NO: 50 - protein, 119 aa, synthetic construct; identical to SEQ ID NO: 24

SEQ ID NO: 51 - protein, 107 aa, synthetic construct
DIVMTQTPLSLSVTPGEPASISCRASQSISDYLYWYLQKPGQSPRRLIFTGSQRASGVPDRFSGSGSGTDFTLRISRVEADDVGVYYCQQGVRFPYTFGPGTKLEIK

SEQ ID NO: 52 - protein, 119 aa, synthetic construct; identical to SEQ ID NO: 24

SEQ ID NO: 53 - protein, 107 aa, synthetic construct
DIVMTQTPLSLSVTPGEPASISCRASQSISDYLYWYLQKPGQSPRLVFNHASPVPSGVPDRFSGSGSGTDFTLRISRVEADDVGVYYCQQGIRFPYTFGPGTKLEIK

SEQ ID NO: 54 - protein, 119 aa, synthetic construct; identical to SEQ ID NO: 26

SEQ ID NO: 55 - protein, 107 aa, synthetic construct
DIVMTQTPLSLSVTPGEPASISCRASQSISDYLYWYLQKPGQSPRLVINWGSQRASGVPDRFSGSGSGTDFTLRISRVEADDVGVYYCQQGAIFPYTFGPGTKLEIK

SEQ ID NO: 56 - protein, 119 aa, synthetic construct
DVQLVESGGDLVKPGGSLRLTCVGSGFTFSNYAMSWVRQAPGEGLQWVATIGSGGHYTYHPDSVKGRFTISRDNAKNTLYLQMNSLQTEDTASYYCTRETDGYGPMDLWGQGTLVTVSS

SEQ ID NO: 57 - protein, 107 aa, synthetic construct
DIVMTQTPLSLSVTPGEPASISCRASQSISDYLYWYLQKPGQSPRRLIYFGSLLSSGVPDRFSGSGSGTDFTLRISRVEADDVGVYYCQQGLLFPYTFGPGTKLEIK

SEQ ID NO: 58 - protein, 119 aa, synthetic construct; identical to SEQ ID NO: 26

SEQ ID NO: 59 - protein, 107 aa, synthetic construct
DIVMTQTPLSLSVTPGEPASISCRASQSISDYLYWYLQKPGQSPRLIFNPLSVRRSGVPDRFSGSGSGTDFTLRISRVEADDVGVYYCQQGYVFPYTFGPGTKLEIK

SEQ ID NO: 60 - protein, 8 aa, synthetic construct
GFTFSNYA

SEQ ID NO: 61 - protein, 8 aa, synthetic construct
IGSGGHYT

SEQ ID NO: 62 - protein, 6 aa, synthetic construct
QSISDY

SEQ ID NO: 63 - protein, 12 aa, synthetic construct
TRETDGYDGMDY

SEQ ID NO: 64 - protein, 12 aa, synthetic construct
TRETDGYDGLDY

SEQ ID NO: 65 - protein, 9 aa, synthetic construct
VFYTDSAEV

SEQ ID NO: 66 - protein, 9 aa, synthetic construct
LIYYASQSI

SEQ ID NO: 67 - protein, 9 aa, synthetic construct
QNGHSFPYT

SEQ ID NO: 68 - protein, 9 aa, synthetic construct
QQGHSFPYT

SEQ ID NO: 69 - protein, 9 aa, synthetic construct
QSGHSFPYT

SEQ ID NO: 70 - protein, 20 aa, synthetic construct
METDTLLLWVLLLWVPGSTG

SEQ ID NO: 71 - protein, 12 aa, synthetic construct
TRETDGYGPMDV

SEQ ID NO: 72 - protein, 13 aa, synthetic construct
VGSGFTFSNYAMS

SEQ ID NO: 73 - protein, 13 aa, synthetic construct
ATIGSGGHYTYHP

SEQ ID NO: 74 - protein, 11 aa, synthetic construct
RASQSISDYLY

SEQ ID NO: 75 - protein, 9 aa, synthetic construct
IFFTESQRA

SEQ ID NO: 76 - protein, 9 aa, synthetic construct
IFFASSLYD

SEQ ID NO: 77 - protein, 9 aa, synthetic construct
IFFGYSGTK

SEQ ID NO: 78 - protein, 9 aa, synthetic construct
VFFTVSGGV

SEQ ID NO: 79 - protein, 9 aa, synthetic construct
VFFTPSDSA

SEQ ID NO: 80 - protein, 12 aa, synthetic construct
TRETDGYGPLDV

SEQ ID NO: 81 - protein, 12 aa, synthetic construct
  VARIANT 8: note = X can be D or G
  VARIANT 9: note = X can be G or P
  VARIANT 10: note = X can be I, L or M
  VARIANT 11: note = X can be D or L
  VARIANT 12: note = X can be L, V or Y
TRETDGYXXXXX

SEQ ID NO: 82 - protein, 9 aa, synthetic construct
  VARIANT 2: note = X can be S, N or Q
  VARIANT 4: note = X can be A, H , I , L , M , P, S, V or Y
  VARIANT 5: note = X can be F, I, L, Q, R, S, or V
  VARIANT 8: note = X can be F or Y
QXGXXFPXT

SEQ ID NO: 83 - protein, 5 aa, synthetic construct
NYAMS

SEQ ID NO: 84 - protein, 7 aa, synthetic construct
GFTFSNY

SEQ ID NO: 85 - protein, 17 aa, synthetic construct
TIGSGGHYTYHPDSVKG

SEQ ID NO: 86 - protein, 6 aa, synthetic construct
GSGGHY

SEQ ID NO: 87 - protein, 10 aa, synthetic construct
ETDGYDGMDY

SEQ ID NO: 88 - protein, 7 aa, synthetic construct
YASQSIS

SEQ ID NO: 89 - protein, 8 aa, synthetic construct
GGGSGGGS

SEQ ID NO: 90 - protein, 107 aa, synthetic construct
DIVMTQTPLSLSVTPGEPASISCRASQSISDYLYWYLQKPGQSPRLIFNPLSVRRSGVPDRFSGSGSGTDFTLRISRVEADDVGVYYCQQGMVFPYTFGPGTKLEIK

SEQ ID NO: 91 - protein, 12 aa, synthetic construct
WYLQKPGQSPRR

SEQ ID NO: 92 - protein, 12 aa, synthetic construct
WYLQKPGQSPRL

SEQ ID NO: 93 - protein, 9 aa, synthetic construct
LFNVLSQRS

SEQ ID NO: 94 - protein, 9 aa, synthetic construct
LFNSVSPTR

SEQ ID NO: 95 - protein, 9 aa, synthetic construct
IINYGSVLL

SEQ ID NO: 96 - protein, 9 aa, synthetic construct
IINPMSGQL

SEQ ID NO: 97 - protein, 9 aa, synthetic construct
LIYFGSVRS

SEQ ID NO: 98 - protein, 9 aa, synthetic construct
IFNPLSVRR

SEQ ID NO: 99 - protein, 9 aa, synthetic construct
IIFTGSQRN

SEQ ID NO: 100 - protein, 9 aa, synthetic construct
IFNHLSPSS

SEQ ID NO: 101 - protein, 9 aa, synthetic construct
VFFHSSYRQ

SEQ ID NO: 102 - protein, 9 aa, synthetic construct
IINQASSKL

SEQ ID NO: 103 - protein, 9 aa, synthetic construct
LFNYGSVLA

SEQ ID NO: 104 - protein, 9 aa, synthetic construct
VIYTGSQRS

SEQ ID NO: 105 - protein, 9 aa, synthetic construct
LIFTGSQRA

SEQ ID NO: 106 - protein, 9 aa, synthetic construct
VFNHASPVP

SEQ ID NO: 107 - protein, 9 aa, synthetic construct
VINWGSQRA

SEQ ID NO: 108 - protein, 9 aa, synthetic construct
LIYFGSLLS

SEQ ID NO: 109 - protein, 9 aa, synthetic construct
QQGLVFPYT

SEQ ID NO: 110 - protein, 9 aa, synthetic construct
QQGVVFPYT

SEQ ID NO: 111 - protein, 9 aa, synthetic construct
QQGLLFPYT

SEQ ID NO: 112 - protein, 9 aa, synthetic construct
QQGLQFPYT

SEQ ID NO: 113 - protein, 9 aa, synthetic construct
QQGMVFPYT

SEQ ID NO: 114 - protein, 9 aa, synthetic construct
QQGAFFPYT

SEQ ID NO: 115 - protein, 9 aa, synthetic construct
QQGSVFPYT

SEQ ID NO: 116 - protein, 9 aa, synthetic construct
QQGMLFPFT

SEQ ID NO: 117 - protein, 9 aa, synthetic construct
QQGPLFPYT

SEQ ID NO: 118 - protein, 9 aa, synthetic construct
QQGYLFPYT

SEQ ID NO: 119 - protein, 9 aa, synthetic construct
QQGVRFPYT

SEQ ID NO: 120 - protein, 9 aa, synthetic construct
QQGIRFPYT

SEQ ID NO: 121 - protein, 9 aa, synthetic construct
QQGAIFPYT

SEQ ID NO: 122 - protein, 9 aa, synthetic construct
QQGYVFPYT

SEQ ID NO: 123 - protein, 12 aa, synthetic construct
TRETDGYGPILD

SEQ ID NO: 124 - protein, 12 aa, synthetic construct
TRETDGYGPMLD

SEQ ID NO: 125 - protein, 12 aa, synthetic construct
TRETDGYGPLDL

SEQ ID NO: 126 - protein, 12 aa, synthetic construct
TRETDGYGPMDL

SEQ ID NO: 127 - protein, 222 aa, synthetic construct
DVQLVESGGDLVKPGGSLRLTCVGSGFTFSNYAMSWVRQAPGEGLQWVATIGSGGHYTYHPDSVKGRFTISRDNAKNTLYLQMNSLQTEDTASYYCTRETDGYDGMDYWGQGTLVTVSSASTTAPSVFPLAPSCGTTSGATVALACLVLGYFPEPVTVSWNSGALTSGVHTFPAVLQASGLYSLSSMVTVPSSRWLSDTFTCNVAHPPSNTKVDKTVRKTDH

SEQ ID NO: 128 - protein, 217 aa, synthetic construct
DIVMTQTPLSLSVTPGEPASISCRASQSISDYLYWYLQKPGQSPRRLIYYASQSISGVPDRFSGSGSGTDFTLRISRVEADDVGVYYCQNGHSFPYTFGPGTKLEIKRSDAQPSVFLFQPSLDELHTGSASIVCILNDFYPKEVNVKWKVDGVVQNKGIQESTTEQNSKDSTYSLSSTLTMSSTEYQSHEKFSCEVTHKSLASTLVKSFNRSECQRE

SEQ ID NO: 129 - protein, 217 aa, synthetic construct
EIQMTQSPSSLSASVGDRVTITCRASQSISDYLYWYQQKPGKAPKLLIYYASQSISGVPSRFSGSGSGTDFTLTISSLQPEDFATYYCQNGHSFPYTFGPGTKLEIKRSDAQPSVFLFQPSLDELHTGSASIVCILNDFYPKEVNVKWKVDGVVQNKGIQESTTEQNSKDSTYSLSSTLTMSSTEYQSHEKFSCEVTHKSLASTLVKSFNRSECQRE

(19) United States
(12) Patent Application Publication
(10) Pub. No.: US 2025/0051435 A1
(43) Pub. Date: Feb. 13, 2025

(54) ANTI-IL-5 ANTIBODIES AND USES THEREOF

(71) Applicant: INVETX, INC., Boston, MA (US)

(72) Inventors: William BRONDYK, Boston, MA (US); Leila SEVIGNY, Boston, MA (US)

(21) Appl. No.: 18/799,544

(22) Filed: Aug. 9, 2024

Related U.S. Application Data
(60) Provisional application No. 63/532,157, filed on Aug. 11, 2023.

(51) Int. Cl. C07K 16/24

(52) U.S. Cl. CPC C07K 16/244; C07K 2317/24; C07K 2317/76; C07K 2317/92

(57) ABSTRACT

The invention provides novel anti-IL-5 proteins, antibodies and IL-5 binding fragments thereof, which inhibit association of IL-5 with IL-5 receptor and are suitable for administration to a human or feline subject. The invention provides novel compositions and methods of treating, alleviating the symptoms of, or preventing, lung diseases, cardiovascular diseases, cancers, metabolic diseases, neurological diseases, and infectious diseases, comprising administering an effective amount of an anti-IL-5 protein, antibody, or fragment thereof. The methods and compositions are used to treat or prevent IL-5-related disorders.

### INCORPORATION BY REFERENCE

This application claims priority to U.S. provisional application Ser. No. 63/532,157, filed Aug. 11, 2023, which is incorporated by reference herein in its entirety.

All documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention. More specifically, all referenced documents are incorporated by reference to the same extent as if each individual document was specifically and individually indicated to be incorporated by reference.

### SEQUENCE LISTING

This application as originally filed includes/contains a Sequence Listing filed in electronic form in eXtensible Markup Language (XML) format entitled Y9432-99004.xml, created on Aug. 29, 2024 and having a size of 123,249 bytes. The contents of the Sequence Listing are incorporated herein in its entirety.

### FIELD OF THE INVENTION

The invention provides novel anti-IL-5 proteins and antibodies that are suitable for administration to a human or feline subject. The invention also provides novel compositions and methods of treating asthma or eliciting an antiasthmatic or antiallergenic effect in a human or feline subject, comprising administering an effective amount of an anti-IL-5 protein, antibody or fragment thereof. The methods and compositions are used to treat or prevent IL-5-related disorders.

### BACKGROUND OF THE INVENTION

Interleukin 5 (IL-5) is an interleukin produced by type-2 T helper cells and mast cells. Through binding to the IL-5 receptor, IL-5 stimulates B cell growth and increases immunoglobulin secretion-primarily IgA. It is also a key modulator in eosinophil activation.

IL-5 is a 115 amino acid (in human, 133 amino acids in mouse) long TH2 cytokine that is part of the hematopoietic family. Unlike other members of this cytokine family (namely interleukin 3 and CM-CSF), this glycoprotein in its active form is a homodimer.

The IL-5 has long been associated with the cause of several allergic diseases including allergic rhinitis and asthma, wherein a large increase in the number of circulating, airway tissue, and induced sputum eosinophils have been observed.

Citation or identification of any document in this application is not an admission that such document is available as prior art to the present invention.

### SUMMARY OF THE INVENTION

The invention provides an antigen binding protein that specifically binds to interleukin-5 (IL-5). In certain embodiments, that IL-5 binding protein comprises: (a) a heavy chain complementarity determining region 1 (HCDR1) comprising X₁X₂X₃X₄X₅X₆X₇X₈, wherein X₁ comprises A, G, I, L, M, W, F, P, or V, X₂ comprises A, G, I, L, M, W, F, P, or V, X₃ comprises C, S, T, Y, N, or Q, X₄ comprises A, G, I, L, M, W, F, P, or V, X₅ comprises A, G, I, L, M, W, F, P, or V, X₆ comprises H, K, or R, X₇ comprises C, S, T, Y, N, or Q, and X₈ comprises C, S, T, Y, N, or Q; (b) a heavy chain complementarity determining region 2 (HCDR2) comprising X₁X₂X₃X₄X₅X₆X₇X₈, wherein X₁ comprises A, G, I, L, M, W, F, P, or V, X₂ comprises A, G, I, L, M, W, F, P, or V, X₃ comprises C, S, T, Y, N, or Q, X₄ comprises A, G, I, L, M, W, F, P, or V, X₅ comprises A, G, I, L, M, W, F, P, or V, X₆ comprises H, K, or R, X₇ comprises C, S, T, Y, N, or Q, and X₈ comprises a C, S, T, Y, N, or Q; (c) a heavy chain complementarity determining region 3 (HCDR3) comprising X₁X₂X₃X₄X₅X₆X₇X₈X₉X₁₀X₁₁X₁₂, wherein X₁ comprises C, S, T, Y, N, or Q, X₂ comprises H, K, or R, X₃ comprises an E or D, X₄ comprises C, S, T, Y, N, or Q, X₅ comprises E or D, X₆ comprises A, G, I, L, M, W, F, P, or V, X₇ comprises C, S, T, Y, N, or Q, X₈ comprises D, E, or G, X₉ comprises A, G, I, L, M, W, F, P, or V, X₁₀ comprises A, G, I, L, M, W, F, P, or V, X₁₁ comprises D, E, or L, and X₁₂ comprises C, L, S, T, V, Y, N, or Q; (d) a light chain complementarity determining region 1 (LCDR1) comprising X₁X₂X₃X₄X₅X₆, wherein X₁ comprises C, S, T, Y, N, or Q, X₂ comprises C, S, T, Y, N, or Q, X₃ comprises A, G, I, L, M, W, F, P, or V, X₄ comprises C, S, T, Y, N, or Q, X₅ comprises E or D, and X₆ comprises C, S, T, Y, N, or Q; (e) a light chain complementarity determining region 2 (LCDR2) comprising X₁X₂X₃X₄X₅X₆X₇X₈X₉X₁₀, wherein X₁ comprises L or R, X₂ comprises A, G, I, L, M, W, F, P, or V, X₂ comprises A, G, I, L, M, W, F, P, or V, X₄ comprises C, S, T, Y, N, or Q, X₅ comprises C, S, T, Y, N, or Q, X₆ comprises D, E, A, G, I, L, M, W, F, P, or V, X₇ comprises C, S, T, Y, N, or Q, X₈ comprises C, S, T, Y, N, Q, A, G, I, L, M, W, F, P, or V, X₉ comprises D, E, C, S, T, Y, N, or Q, X₁₀ comprises A, G, I, L, M, W, F, P, Y or V; and (f) a light chain complementarity determining region 3 (LCDR3) comprising X₁X₂X₃X₄X₅X₆X₇X₈X₉, wherein X₁ comprises C, S, T, Y, N, or Q, X₂ comprises C, S, T, Y, N, or Q, X₃ comprises A, G, I, L, M, W, F, P, or V, X₄ comprises A, H, I, K, L, M, P, R, S, V, or Y, X₅ comprises C, F, I, L, R, S, T, Y, N, or Q, X₆ comprises A, G, I, L, M, W, F, P, or V, X₇ comprises A, G, I, L, M, W, F, P, or V, X₈ comprises C, S, T, F, Y, N, or Q, and X₉ comprises C, S, T, Y, N, or Q.

In certain embodiments, the IL-5 binding protein comprises (a) HCDR1 comprising GFTFSNYA (SEQ ID NO:60) or differing at no more than one or two positions; and/or (b) HCDR2 comprising IGSGGHYT (SEQ ID NO:61) or differing at no more than one or two positions; and/or (c) HCDR3 comprising TRETDGYX₈X₉X₁₀X₁₁X₁₂, (SEQ ID NO:81) wherein X₈ comprises D or G, X₉ comprises G or P, X₁₀ comprises I, L, or M, X₁₁ comprises D or L, and X₁₂ comprises L, V, or Y; and/or (d) LCDR1 comprising QSISDY (SEQ ID NO:62) or differing at no more than one or two positions; and/or (e) LCDR2 comprising X₁X₂X₃X₄X₅X₆SX₈X₉X₁₀, wherein X₈ comprises A, F, G, H, P, Q, S, T, V, or Y, and X₆ comprises A, D, E, G, L, M, P, S, V, or Y; and/or (f) LCDR3 comprising QX₂GX₄X₅FPX₈T (SEQ ID NO:82), wherein X₂ comprises S, N, or Q, X₄ comprises A, H, I, L, M, P, S, V, or Y, X₅ comprises F, I, L, Q, R, S, or V, X₅ comprises F or Y.

In certain embodiments, the IL-5 binding protein comprises LCDR2, wherein X₁ comprises L or R, X₂ comprises I, L, or V, X₃ comprises I or F, X₄ comprises F, K, N, or Y, X₈ comprises A, F, G, H, P, Q, S, T, V, or Y, X₆ comprises A, D, E, G, L, M, P, S, V, or Y, X₈ comprises A, D, G, L. P, Q, S, V, or Y, X₉ comprises E, G, K, L, Q, R, S, T, of Y, and X₁₀ comprises A, D, I, L, K, N, Q, R, S, or V.

In certain embodiments, the IL-5 binding protein comprises no more than two (2) substitutions per CDR as compared to an HCDR1, HCDR2, and HCDR3 depicted in FIG. 3 and an LCDR1, LCDR2, and LCDR3 depicted in FIG. 4.

In certain embodiments, the IL-5 binding protein comprises no more than one (1) substitution per CDR as compared to an HCDR1, HCDR2, and HCDR3 depicted in FIG. 3 and an LCDR1, LCDR2, and LCDR3 depicted in FIG. 4.

In certain embodiments, the IL-5 binding protein comprises one or more HCDRs of any one of SEQ ID NO:3, SEQ ID NO:5, SEQ ID NO:8, SEQ ID NO:11, SEQ ID NO:19, SEQ ID NO:20, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56 or SEQ ID NO:58 and one or more LCDRs of SEQ ID NO:4, SEQ ID NO:6, SEQ ID NO:7, SEQ ID NO:9, SEQ ID NO:10, SEQ ID NO:12, SEQ ID NO:13, SEQ ID NO:14, SEQ ID NO:15, SEQ ID NO:16, SEQ ID NO:17, SEQ ID NO:18, SEQ ID NO:21, SEQ ID NO:22, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, or SEQ ID NO:59.

In certain embodiments, the IL-5 binding protein comprises the HCDRs of any one of SEQ ID NO:3, SEQ ID NO:5, SEQ ID NO:8, SEQ ID NO:11, SEQ ID NO:19, SEQ ID NO:20, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56 or SEQ ID NO:58 and the LCDRs of SEQ ID NO:4, SEQ ID NO:6, SEQ ID NO:7, SEQ ID NO:9, SEQ ID NO:10, SEQ ID NO:12, SEQ ID NO:13, SEQ ID NO:14, SEQ ID NO:15, SEQ ID NO:16, SEQ ID NO:17, SEQ ID NO:18, SEQ ID NO:21, SEQ ID NO:22, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, or SEQ ID NO:59.

In certain embodiments, the IL-5 binding protein comprises the HCDRs of SEQ ID NO:20 and LCDRs of SEQ ID NO:23, the HCDRs of SEQ ID NO:19 and LCDRs of SEQ ID NO:21, the HCDRs of SEQ ID NO:19 and LCDRs of SEQ ID NO:22, the HCDRs of SEQ ID NO:19 and LCDRs of SEQ ID NO:23, the HCDRs of SEQ ID NO:20 and LCDRs of SEQ ID NO:21, or the HCDRs of SEQ ID NO:20 and LCDRs of SEQ ID NO:22.

In certain embodiments, the IL-5 binding protein comprises the HCDRs of SEQ ID NO:24 and LCDRs of SEQ ID NO:25, the HCDRs of SEQ ID NO:26 and LCDRs of SEQ ID NO:27, the HCDRs of SEQ ID NO:28 and LCDRs of SEQ ID NO:29, the HCDRs of SEQ ID NO:30 and LCDRs of SEQ ID NO:31, the HCDRs of SEQ ID NO:32 and LCDRs of SEQ ID NO:33, the HCDRs of SEQ ID NO:34 and LCDRs of SEQ ID NO:35, the HCDRs of SEQ ID NO:36 and LCDRs of SEQ ID NO:37, the HCDRs of SEQ ID NO:38 and LCDRs of SEQ ID NO:39, the HCDRs of SEQ ID NO:40 and LCDRs of SEQ ID NO:41, the HCDRs of SEQ ID NO:42 and LCDRs of SEQ ID NO:43, the HCDRs of SEQ ID NO:44 and LCDRs of SEQ ID NO:45, the HCDRs of SEQ ID NO:46 and LCDRs of SEQ ID NO:47, the HCDRs of SEQ ID NO:48 and LCDRs of SEQ ID NO:49, the HCDRs of SEQ ID NO:50 and LCDRs of SEQ ID NO:51, the HCDRs of SEQ ID NO:52 and LCDRs of SEQ ID NO:53, the HCDRs of SEQ ID NO:54 and LCDRs of SEQ ID NO:55, the HCDRs of SEQ ID NO:56 and LCDRs of SEQ ID NO:57, or the HCDRs of SEQ ID NO:58 and LCDRs of SEQ ID NO:59.

In certain embodiments, the IL-5 binding protein comprises the HCDRs of SEQ ID NO:8 and the LCDRs of SEQ ID NO:7, the HCDRs of SEQ ID NO:11 and the LCDRs of SEQ ID NO:7, the HCDRs of SEQ ID NO:5 and the LCDRs of SEQ ID NO:9, the HCDRs of SEQ ID NO:5 and the LCDRs of SEQ ID NO:10, the HCDRs of SEQ ID NO:5 and the LCDRs of SEQ ID NO:12, the HCDRs of SEQ ID NO:5 and the LCDRs of SEQ ID NO:13, the HCDRs of SEQ ID NO:5 and the LCDRs of SEQ ID NO:14, the HCDRs of SEQ ID NO:5 and the LCDRs of SEQ ID NO:15, the HCDRs of SEQ ID NO:5 and the LCDRs of SEQ ID NO:16, the HCDRs of SEQ ID NO:5 and the LCDRs of SEQ ID NO:17, or the HCDRs of SEQ ID NO:5 and the LCDRs of SEQ ID NO:18.

In certain embodiments, the IL-5 binding protein comprises a heavy chain framework (FR1H+FR2H+FR3H+FR4H) at least 75%, or at least 80%, or at least 85%, or at least 90%, or at least 93%, or at least 95% identical, or identical to SEQ ID NO:3, SEQ ID NO:5, SEQ ID NO:8, SEQ ID NO:11, SEQ ID NO:19, SEQ ID NO:20, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56 or SEQ ID NO:58.

In certain embodiments, the IL-5 binding protein comprises a light chain framework (FR1L+FR2L+FR3L+FR4L) at least 75%, or at least 80%, or at least 85%, or at least 90%, or at least 93%, or at least 95% identical, or identical to SEQ ID NO:4, SEQ ID NO:6, SEQ ID NO:7, SEQ ID NO:9, SEQ ID NO:10, SEQ ID NO:12, SEQ ID NO:13, SEQ ID NO:14, SEQ ID NO:15, SEQ ID NO:16, SEQ ID NO:17, SEQ ID NO:18, SEQ ID NO:21, SEQ ID NO:22, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, or SEQ ID NO:59.

In certain embodiments, the IL-5 binding protein comprises a VH domain at least 80%, or at least 85%, or at least 90%, or at least 93%, or at least 95% identical, or at least 97% identical, or identical to SEQ ID NO:3, SEQ ID NO:5, SEQ ID NO:8, SEQ ID NO:11, SEQ ID NO:19, SEQ ID NO:20, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56 or SEQ ID NO:58.

In certain embodiments, the IL-5 binding protein comprises a VL domain at least 80%, or at least 85%, or at least 90%, or at least 93%, or at least 95% identical, or at least 97% identical, or identical to SEQ ID NO:4, SEQ ID NO:6, SEQ ID NO:7, SEQ ID NO:9, SEQ ID NO:10, SEQ ID NO:12, SEQ ID NO:13, SEQ ID NO:14, SEQ ID NO:15, SEQ ID NO:16, SEQ ID NO:17, SEQ ID NO:18, SEQ ID NO:21, SEQ ID NO:22, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, or SEQ ID NO:59.

In certain embodiments, the antigen binding protein comprises feline or felinized frameworks. In certain embodiments, the antigen binding protein comprises canine or caninized frameworks. In certain embodiments, the antigen binding protein comprises human or humanized frameworks.

In an aspect, the invention provides an isolated nucleic acid sequence encoding any one of the aforementioned anti-IL-5 antibodies or antibody fragments, and a vector comprising or capable of expressing any one of the anti-IL-5 antibodies or antibody fragments.

In another aspect, the invention provides a recombinant cell which comprises a nucleic acid sequence encoding any one of the aforementioned anti-IL-5 antibodies or antibody fragments, or a vector comprising or capable of expressing any one of the anti-IL-5 antibodies or antibody fragments.

The invention provides a method of producing any one of the aforementioned anti-IL-5 antibodies or antibody fragments, which comprises culturing the cell capable of expressing the anti-IL-5 antibody or antibody fragment under conditions that result in production of the antibody or antibody fragment.

The invention provides a pharmaceutical composition comprising a therapeutically effective amount of any one of the aforementioned anti-IL-5 antibodies or antibody fragments.

In an aspect, the invention provides a method of suppressing IL-5 mediated activation of an eosinophil, which comprises culturing the eosinophil in the presence of any one of the aforementioned anti-IL-5 antibodies or antibody fragments.

In an aspect, the invention provides a method of suppressing an eosinophil mediated inflammatory response in a subject which comprise administering to the subject a therapeutically effective amount of any one of the aforementioned anti-IL-5 antibodies or antibody fragments.

In an aspect, the invention provides a method of inhibiting binding of IL-5 to IL-5 receptor in a subject, which comprises administering to the subject a therapeutically effective amount of any one of the aforementioned anti-IL-5 antibodies or antibody fragments.

In an aspect, the invention provides a method of detecting IL-5 in a sample comprising incubating the sample with any one of the aforementioned anti-IL-5 antibodies or antibody fragments and detecting the anti IL-5 antibody or antibody fragment bound to IL-5 in the sample.

Accordingly, it is an object of the invention not to encompass within the invention any previously known product, process of making the product, or method of using the product such that Applicants reserve the right and hereby disclose a disclaimer of any previously known product, process, or method. It is further noted that the invention does not intend to encompass within the scope of the invention any product, process, or making of the product or method of using the product, which does not meet the written description and enablement requirements of the USPTO (35 U.S.C. § 112, first paragraph) or the EPO (Article 83 of the EPC), such that Applicants reserve the right and hereby disclose a disclaimer of any previously described product, process of making the product, or method of using the product. It may be advantageous in the practice of the invention to be in compliance with Art. 53(c) EPC and Rule 28(b) and (c) EPC. All rights to explicitly disclaim any embodiments that are the subject of any granted patent(s) of applicant in the lineage of this application or in any other lineage or in any prior filed application of any third party is explicitly reserved. Nothing herein is to be construed as a promise.

It is noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

These and other embodiments are disclosed or are obvious from and encompassed by the following Detailed Description.

### BRIEF DESCRIPTION OF THE DRAWINGS

The following detailed description, given by way of example, but not intended to limit the invention solely to the specific embodiments described, may best be understood in conjunction with the accompanying drawings.

FIG. 1A depicts the amino acid sequence of an IL-5 receptor α (IL-5Rα) construct comprising: Signal sequence—extracellular domain (ECD) of the feline IL-5 receptor α—V5 epitope—TEV protease site—human IgG1 Fc (SEQ ID NO:1).

FIG. 1B depicts the amino acid sequence of an IL-5Rα construct comprising: Signal sequence—extracellular domain (ECD) of the feline IL-5 receptor α—2X (Gly-Gly-Gly-Ser) (SEQ ID NO:89)—human IgG1—AviTag (SEQ ID NO:2).

FIG. 2 depicts the amino acid sequence of V_{H}C_{H}1 (FIG. 2A) (SEQ ID NO:127) and V_{L}C_{L} portions of the MtxA (FIG. 2B) (SEQ ID NO:129) and MtxB (FIG. 2C) (SEQ ID NO:128) antibodies described herein. The MtxA antibody and MtxB antibody share the same V_{H} (FIG. 2A) (SEQ ID NO: 127) domain, including V_{H} CDRs of murine anti-feline IL-5 antibody 154. The MtxA V_{L} domain (FIG. 2B) (SEQ ID NO:129) and MtxB V_{L} domain (FIG. 2C) (SEQ ID NO:128) share the same V_{L} CDRs of murine anti-feline IL-5 antibody 154 but have different FRs. IMGT CDRs are in bolded text. In MtxA and MtxB clones there is a potential deamidation site (NG) in LCDR3 (boxed in 2B and 2C) and a potential oxidation site (M) in HCDR3 (boxed in 2A).

FIG. 3 (SEQ ID NO:3, 5, 8, 11, 19, 20, 24, 26, 28, 30, 32, 35, 36, 38, 40, 42, 44, 46, 48, 50, 52, 54, 56 and 58) depicts a sequence alignment of V_{H} domains of binding proteins of the invention.

FIG. 4 (SEQ ID NO:4, 6, 7, 9, 10, 12-18, 21-23, 25, 27, 29, 31, 33, 37, 39, 41, 43, 45, 47, 49, 51, 53, 55, 57, 59 and 90) depicts a sequence alignment of V_{L} domains of binding proteins of the invention.

### DETAILED DESCRIPTION OF THE INVENTION

According to certain exemplary embodiments of the present invention, the IL-5 binding protein is an anti-IL-5 antibody or antigen-binding fragment thereof. The term “antibody,” as used herein, includes immunoglobulin molecules comprising four polypeptide chains, two heavy (H) chains and two light (L) chains inter-connected by disulfide bonds, as well as multimers thereof (e.g., IgM). In a typical antibody, each heavy chain comprises a heavy chain variable region (abbreviated herein as HCVR or VH) and a heavy chain constant region. The heavy chain constant region comprises three domains, CH1, CH2 and CH3. Each light chain comprises a light chain variable region (abbreviated herein as LCVR or VL) and a light chain constant region. The light chain constant region comprises one domain (CL). The VH and VL regions can be further subdivided into regions of hypervariability, termed complementarity determining regions (CDRs), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4. In different embodiments of the invention, the FRs of the antibody (or antigen-binding portion thereof) may be identical to the feline germline sequences, or may be naturally or artificially modified. An amino acid consensus sequence may be defined based on a side-by-side analysis of two or more CDRs.

Antibody residues that have a substantial impact on affinity and specificity of binding to target antigen are primarily located in CDRs. Kabat et al. compiled and aligned immunoglobulin heavy and light chain sequences and were the first to propose a standardized numbering scheme for the variable regions of immunoglobulins identifying conserved and hypervariable regions and residues. (Kabat E A et al., 1979, Sequences of Immunoglobulin Chains: Tabulation and Analysis of Amino Acid Sequences of Precursors, V-regions, C-regions, J-Chain and BP-Microglobulins, Department of Health, Education, and Welfare, Public Health Service, National Institutes of Health). While the Kabat system is a widely adopted standard for numbering antibody residues, the hypervariable regions defined by Kabat do not exactly match with the structural aspects of antigen-binding loops. Chothia and Lesk developed a structure-based numbering scheme by aligning crystal structures of antibody variable regions and classified CDR loops in a small number of “canonical” classes (Chothia C, et al., 1987, Canonical structures for the hypervariable regions of immunoglobulins. J. Mol. Biol. 196:901-17. doi: 10.1016/0022-2836(87)90412-8). An advantage of the Chothia numbering scheme is that topologically aligned residues from different antibodies are localized at the same position number and the Chothia CDR definition corresponds in most antibody sequences to the structural antigen-binding loop. Lefranc introduced a new system based on germ-line sequences intended to standardize numbering for all proteins of the immunoglobulin superfamily, including T cell receptor chains. (Giudicelli V et al., 1997, IMGT, the international ImMunoGeneTics database. Nucleic Acids Res. 25:206-11), which was then extended to entire variable domains (Lefranc M-P et al., 2003, IMGT unique numbering for immunoglobulin and T cell receptor variable domains and Ig superfamily V-like domains. Dev Comp Immunol. 27:55-77. doi: 10.1016/S0145-305X(02)00039-3). Additional numbering systems have been proposed to align unconventional frameworks (Abhinandan K R et al., 2008, Analysis and improvements to Kabat and structurally correct numbering of antibody variable domains. Mol Immunol. 45:3832-9. doi: 10.1016/j.molimm.2008.05.022) and to subdivide variable chain sequences into multiple fragments including structurally invariant “cores” (Gelfand et al., 1998, Algorithmic determination of core positions in the VL and VH domains of immunoglobulin molecules. J Comput Biol. (1998) 5:467-77). In certain embodiments of the invention, CDR residues are identified according to such a standard system as set forth above. In certain embodiments, antibodies of the invention are identified by all or a subset of Kabat CDR residues of the antibody sequences set forth herein. In certain embodiments, antibodies of the invention are identified by all or a subset of Chothia CDR residues of the antibody sequences set forth herein. In certain embodiments, antibodies of the invention are identified by all or a subset of IMGT CDR residues of the antibody sequences set forth herein. In certain embodiments, antibodies of the invention are identified by CDR residues defined by two or more systems, comprising e.g., but not limited to, all or a subset of residues of HCDR1 according to Kabat, all or a subset of residues of HCDR2 according to Chothia, all or a subset of residues of HCDR3 according to Kabat, all or a subset of residues of LCDR1 according to Kabat, all or a subset of residues of LCDR2 according to IMGT, and all or a subset of residues of LCDR3 according to Chothia.

For reference, the following table shows relative locations of Kabat, Chothia, and IMGT CDRs mapped on murine antibody 154 VH (SEQ ID NO:3) and VL (SEQ ID NO:4) described herein that binds to feline IL-5. “XXX” indicates CDR regions indicted herein as X₁X₂X₃X₄X₅X₆X₇X₈ for HCDR1, X₁X₂X₃X₄X₅X₆X₇X₈ for HCDR2, X₁X₂X₃X₄X₅X₆X₇X₈X₉X₁₀X₁₁X₁₂ for HCDR3, X₁X₂X₃X₄X₅X₆ for LCR1, X₁X₂X₃X₄X₅X₆X₇X₈X₉X₁₀ for LCDR2, and X₁X₂X₃X₄X₅X₆X₇X₈X₉ for LCDR3.

| | HCDR1 | HCDR2 | HCDR3 |
|---|---|---|---|
| Kabat | NYAMS | TIGSGGHYTYHPDSVKG | ETDGYDGMDY |
| | (SEQ ID NO: 83) | (SEQ ID NO: 85) | (SEQ ID NO: 87) |
| Chothia | GFTFSNY | GSGGHY | ETDGYDGMDY |
| | (SEQ ID NO: 84) | (SEQ ID NO: 86) | SEQ ID NO: 87) |
| IMGT | GFTFSNYA | IGSGGHYT | TRETDGYDGMDY |
| | (SEQ ID NO: 60) | (SEQ ID NO: 61) | (SEQ ID NO: 63) |
| | XXXXXXXX | XXXXXXXX | XXXXXXXXXXXX |
| | LCDR1 | LCDR2 | LCDR3 |
| Kabat | RASQSISDYLY | YASQSIS | QNGHSFPYT |
| | (SEQ ID NO: 74) | (SEQ ID NO: 88) | (SEQ ID NO: 67) |
| Chothia | RASQSISDYLY | YASQSIS | QNGHSFPYT |
| | (SEQ ID NO: 74) | (SEQ ID NO: 88) | (SEQ ID NO: 67) |
| IMGT | QSISDY | YAS | QNGHSFPYT |
| | (SEQ ID NO: 62). | | (SEQ ID NO: 67) |
| | XXXXXX | XXXXXXXXXX | XXXXXXXXX |

In one aspect, the invention provides a binding protein suitable for use in a mammal, for example, but without limitation, a feline. In certain embodiments, a felinized anti-IL-5 binding protein comprises a heavy chain complementarity determining region 1 (HCDR1), a heavy chain complementarity determining region 2 (HCDR2), a heavy chain complementarity determining region 3 (HCDR3), a light chain complementarity determining region 1 (LCDR1), a light chain complementarity region 2 (LCDR2), and a light chain complementarity region 3 (LCDR3).

In some embodiments, the heavy chain complementarity determining region 1 (HCDR1) comprising 3-25 amino acids in length. In some embodiments, the HCDR1 is 5-15 amino acids in length. In some embodiments, the HCDR1 is 6-10 amino acids in length. In some embodiments the HCDR1 is 3 amino acids in length. In some embodiments the HCDR1 is 4 amino acids in length. In some embodiments the HCDR1 is 5 amino acids in length. In some embodiments the HCDR1 is 6 amino acids in length. In some embodiments the HCDR1 is 7 amino acids in length. In some embodiments the HCDR1 is 8 amino acids in length. In some embodiments the HCDR1 is 9 amino acids in length. In some embodiments the HCDR1 is 10 amino acids in length. In some embodiments the HCDR1 is 11 amino acids in length. In some embodiments the HCDR1 is 12 amino acids in length. In some embodiments the HCDR1 is 13 amino acids in length. In some embodiments the HCDR1 is 14 amino acids in length. In some embodiments the HCDR1 is 15 amino acids in length. In some embodiments the HCDR1 is 16 amino acids in length. In some embodiments the HCDR1 is 17 amino acids in length. In some embodiments the HCDR1 is 18 amino acids in length. In some embodiments the HCDR1 is 19 amino acids in length. In some embodiments the HCDR1 is 20 amino acids in length. In some embodiments the HCDR1 is 21 amino acids in length. In some embodiments the HCDR1 is 22 amino acids in length. In some embodiments the HCDR1 is 23 amino acids in length. In some embodiments the HCDR1 is 24 amino acids in length. In some embodiments the HCDR1 is 25 amino acids in length. In some embodiments the HCDR1 comprises the amino acid sequence X₁X₂X₃X₄X₅X₆X₇X₈, wherein X₁ comprises a nonpolar amino acid, wherein X₂ comprises a nonpolar amino acid, wherein X₃ comprises a polar amino acid, wherein X₄ comprises a nonpolar amino acid, wherein X₅ comprises a polar amino acid, wherein X₆ comprises a polar amino acid, wherein X₇ comprises a polar amino acid, and wherein X₈ comprises a nonpolar amino acid. In some embodiments, X₁ comprises an A, G, I, L, M, W, F, P, or V, X₂ comprises an A, G, I, L, M, W, F, P, or V, X₃ comprises a C, S, T, Y, N, or Q, X₄ comprises an A, G, I, L, M, W, F, P, or V, X₅ comprises a C, S, T, Y, N, or Q, X₆ comprises a C, S, T, Y, N, or Q, X₇ comprises a C, S, T, Y, N, or Q, and X₈ comprises an A, G, I, L, M, W, F, P, or V. In some embodiments, X₁ comprises a G, X₂ comprises an F, X₃ comprises a T, X₄ comprises an F, X₅ comprises an S, X₆ comprises an N, X₇ comprises a Y, and X₈ comprises an A. In some embodiments the HCDR1 comprises the amino acid sequence GFTFSNYA (SEQ ID NO:60).

In some embodiments, the heavy chain complementarity determining region 2 (HCDR2) comprising 3-25 amino acids in length. In some embodiments, the HCDR2 is 5-15 amino acids in length. In some embodiments, the HCDR2 is 6-10 amino acids in length. In some embodiments the HCDR2 is 3 amino acids in length. In some embodiments the HCDR2 is 4 amino acids in length. In some embodiments the HCDR2 is 5 amino acids in length. In some embodiments the HCDR2 is 6 amino acids in length. In some embodiments the HCDR2 is 7 amino acids in length. In some embodiments the HCDR2 is 8 amino acids in length. In some embodiments the HCDR2 is 9 amino acids in length. In some embodiments the HCDR2 is 10 amino acids in length. In some embodiments the HCDR2 is 11 amino acids in length. In some embodiments the HCDR2 is 12 amino acids in length. In some embodiments the HCDR2 is 13 amino acids in length. In some embodiments the HCDR2 is 14 amino acids in length. In some embodiments the HCDR2 is 15 amino acids in length. In some embodiments the HCDR2 is 16 amino acids in length. In some embodiments the HCDR2 is 17 amino acids in length. In some embodiments the HCDR2 is 18 amino acids in length. In some embodiments the HCDR2 is 19 amino acids in length. In some embodiments the HCDR2 is 20 amino acids in length. In some embodiments the HCDR2 is 21 amino acids in length. In some embodiments the HCDR2 is 22 amino acids in length. In some embodiments the HCDR2 is 23 amino acids in length. In some embodiments the HCDR2 is 24 amino acids in length. In some embodiments the HCDR2 is 25 amino acids in length. In some embodiments the HCDR2 comprises the amino acid sequence X₁X₂X₃X₄X₅X₆X₇X₈, wherein X₁ comprises a nonpolar amino acid, wherein X₂ comprises a nonpolar amino acid, wherein X₃ comprises a polar amino acid, wherein X₄ comprises a nonpolar amino acid, wherein X₅ comprises a nonpolar amino acid, wherein X₆ comprises a basic amino acid, wherein X₇ comprises a polar amino acid, and wherein X₈ comprises a polar amino acid. In some embodiments, X₁ comprises an A, G, I, L, M, W, F, P, or V, X₂ comprises an A, G, I, L, M, W, F, P, or V, X₃ comprises a C, S, T, Y, N, or Q, X₄ comprises an A, G, I, L, M, W, F, P, or V, X₅ comprises an A, G, I, L, M, W, F, P, or V, X₆ comprises an H, K, or R, X₇ comprises a C, S, T, Y, N, or Q, and X₈ comprises a a C, S, T, Y, N, or Q. In some embodiments, X₁ comprises an I, X₂ comprises a G, X₃ comprises an S, X₄ comprises a G, X₅ comprises a G, X₆ comprises an H, X₇ comprises a Y, and X₈ comprises a T. In some embodiments the HCDR2 comprises the amino acid sequence IGSGGHYT (SEQ ID NO:61).

In some embodiments, the heavy chain complementarity determining region 3 (HCDR3) comprising 3-25 amino acids in length. In some embodiments, the HCDR3 is 5-15 amino acids in length. In some embodiments, the HCDR3 is 6-10 amino acids in length. In some embodiments the HCDR3 is 3 amino acids in length. In some embodiments the HCDR3 is 4 amino acids in length. In some embodiments the HCDR3 is 5 amino acids in length. In some embodiments the HCDR3 is 6 amino acids in length. In some embodiments the HCDR3 is 7 amino acids in length. In some embodiments the HCDR3 is 8 amino acids in length. In some embodiments the HCDR3 is 9 amino acids in length. In some embodiments the HCDR3 is 10 amino acids in length. In some embodiments the HCDR3 is 11 amino acids in length. In some embodiments the HCDR3 is 12 amino acids in length. In some embodiments the HCDR3 is 13 amino acids in length. In some embodiments the HCDR3 is 14 amino acids in length. In some embodiments the HCDR3 is 15 amino acids in length. In some embodiments the HCDR3 is 16 amino acids in length. In some embodiments the HCDR3 is 17 amino acids in length. In some embodiments the HCDR3 is 18 amino acids in length. In some embodiments the HCDR3 is 19 amino acids in length. In some embodiments the HCDR3 is 20 amino acids in length. In some embodiments the HCDR3 is 21 amino acids in length. In some embodiments the HCDR3 is 22 amino acids in length. In some embodiments the HCDR3 is 23 amino acids in length. In some embodiments the HCDR3 is 24 amino acids in length. In some embodiments the HCDR3 is 25 amino acids in length. In some embodiments the HCDR3 comprises the amino acid sequence X₁X₂X₃X₄X₅X₆X₇X₈X₉X₁₀X₁₁X₁₂, wherein X₁ comprises a polar amino acid, wherein X₂ comprises a basic amino acid, wherein X₃ comprises an acidic amino acid, wherein X₄ comprises a polar amino acid, wherein X₅ comprises an acidic amino acid, wherein X₆ comprises a nonpolar amino acid, wherein X₇ comprises a polar amino acid, wherein X₈ comprises an acidic amino acid, wherein X₉ comprises a nonpolar amino acid, wherein X₁₀ comprises a nonpolar amino acid, wherein X₁₁ comprises an acidic amino acid, and wherein X₁₂ comprises a polar amino acid. In some embodiments, X₁ comprises a C, S, T, Y, N, or Q, X₂ comprises an H, K, or R, X₃ comprises an E or D, X₄ comprises a C, S, T, Y, N, or Q, X₈ comprises an E or D, X₆ comprises an A, G, I, L, M, W, F, P, or V, X₇ comprises a C, S, T, Y, N, or Q, X₈ comprises an E or D, X₉ comprises an A, G, I, L, M, W, F, P, or V. X₁₀ comprises an A, G, I, L, M, W, F, P, or V, X₁₁ comprises an E or D, and X₁₂ comprises a C, S, T, Y, N, or Q. In some embodiments, X₁ comprises a T, X₂ comprises an R, X₃ comprises an E, X₄ comprises a T, X₈ comprises a D, X₆ comprises a G, X₇ comprises a Y, X₈ comprises a D, X₉ comprises a G, X₁₀ comprises an M or L, X₁₁ comprises a D, and X₁₂ comprises a Y. In some embodiments, the HCDR3 comprises the amino acid sequence TRETDGYDGMDY (SEQ ID NO:63). In some embodiments, the HCDR3 comprises the amino acid sequence TRETDGYDGLDY (SEQ ID NO:64).

In some embodiments, the light chain complementarity determining region 1 (LCDR1) comprising 3-25 amino acids in length. In some embodiments, the LCDR1 is 5-15 amino acids in length. In some embodiments, the LCDR1 is 6-10 amino acids in length. In some embodiments the LCDR1 is 3 amino acids in length. In some embodiments the LCDR1 is 4 amino acids in length. In some embodiments the LCDR1 is 5 amino acids in length. In some embodiments the LCDR1 is 6 amino acids in length. In some embodiments the LCDR1 is 7 amino acids in length. In some embodiments the LCDR1 is 8 amino acids in length. In some embodiments the LCDR1 is 9 amino acids in length. In some embodiments the LCDR1 is 10 amino acids in length. In some embodiments the LCDR1 is 11 amino acids in length. In some embodiments the LCDR1 is 12 amino acids in length. In some embodiments the LCDR1 is 13 amino acids in length. In some embodiments the LCDR1 is 14 amino acids in length. In some embodiments the LCDR1 is 15 amino acids in length. In some embodiments the LCDR1 is 16 amino acids in length. In some embodiments the LCDR1 is 17 amino acids in length. In some embodiments the LCDR1 is 18 amino acids in length. In some embodiments the LCDR1 is 19 amino acids in length. In some embodiments the LCDR1 is 20 amino acids in length. In some embodiments the LCDR1 is 21 amino acids in length. In some embodiments the LCDR1 is 22 amino acids in length. In some embodiments the LCDR1 is 23 amino acids in length. In some embodiments the LCDR1 is 24 amino acids in length. In some embodiments the LCDR1 is 25 amino acids in length. In some embodiments the LCDR1 comprises the amino acid sequence X₁X₂X₃X₄X₅X₆, wherein X₁ comprises a polar amino acid, wherein X₂ comprises a polar amino acid, wherein X₃ comprises a nonpolar amino acid, wherein X₄ comprises a polar amino acid, wherein X₅ comprises an acidic amino acid, and wherein X₆ comprises a polar amino acid. In some embodiments, X₁ comprises a C, S, T, Y, N, or Q, X₂ comprises a C, S, T, Y, N, or Q, X₃ comprises an A, G, I, L, M, W, F, P, or V X₄ comprises a C, S, T, Y, N, or Q, X₅ comprises an E or D, and X₆ comprises a C, S, T, Y, N, or Q. In some embodiments, X₁ comprises a Q, X₂ comprises an S, X₃ comprises an I, X₄ comprises an S, X₅ comprises a D, and X₆ comprises a Y. In some embodiments, the LCDR1 comprises the amino acid sequence QSISDY (SEQ ID NO:62).

In some embodiments, the light chain complementarity determining region 2 (LCDR2) comprising 3-25 amino acids in length. In some embodiments, the LCDR2 is 5-15 amino acids in length. In some embodiments, the LCDR2 is 6-10 amino acids in length. In some embodiments the LCDR2 is 3 amino acids in length. In some embodiments the LCDR2 is 4 amino acids in length. In some embodiments the LCDR2 is 5 amino acids in length. In some embodiments the LCDR2 is 6 amino acids in length. In some embodiments the LCDR2 is 7 amino acids in length. In some embodiments the LCDR2 is 8 amino acids in length. In some embodiments the LCDR2 is 9 amino acids in length. In some embodiments the LCDR2 is 10 amino acids in length. In some embodiments the LCDR2 is 11 amino acids in length. In some embodiments the LCDR2 is 12 amino acids in length. In some embodiments the LCDR2 is 13 amino acids in length. In some embodiments the LCDR2 is 14 amino acids in length. In some embodiments the LCDR2 is 15 amino acids in length. In some embodiments the LCDR2 is 16 amino acids in length. In some embodiments the LCDR2 is 17 amino acids in length. In some embodiments the LCDR2 is 18 amino acids in length. In some embodiments the LCDR2 is 19 amino acids in length. In some embodiments the LCDR2 is 20 amino acids in length. In some embodiments the LCDR2 is 21 amino acids in length. In some embodiments the LCDR2 is 22 amino acids in length. In some embodiments the LCDR2 is 23 amino acids in length. In some embodiments the LCDR2 is 24 amino acids in length. In some embodiments the LCDR2 is 25 amino acids in length. In some embodiments the LCDR2 comprises the amino acid sequence X₁X₂X₃, wherein X₁ comprises a polar amino acid, wherein X₂ comprises a nonpolar amino acid, and wherein X₃ comprises a polar amino acid. In some embodiments, X₁ comprises a C, S, T, Y, N, or Q, X₂ comprises an A, G, I, L, M, W, F, P, or V, and X₃ comprises a C, S, T, Y, N, or Q. In some embodiments, X₁ comprises a Y, X₂ comprises an A, and X₃ comprises an S. In some embodiments, the LCDR2 comprises the amino acid sequence YAS. In some embodiments the LCDR2 comprises the amino acid sequence X₁X₂X₃X₄X₅X₆X₇X₈X₉, wherein X₁ comprises a nonpolar amino acid, wherein X₂ comprises a nonpolar amino acid, wherein X₃ comprises a polar amino acid, wherein X₄ comprises a polar amino acid, wherein X₅ comprises an acidic or nonpolar amino acid, wherein X₆ comprises a polar amino acid, wherein X₇ comprises a nonpolar or polar amino acid, wherein X₈ comprises an acidic or polar amino acid, and wherein X₉ comprises a nonpolar amino acid. In some embodiments, X₁ comprises an A, G, I, L, M, W, F, P, or V, X₂ comprises an A, G, I, L, M, W, F, P, or V, X₃ comprises a C, S, T, Y, N, or Q, X₄ comprises a C, S, T, Y, N, or Q, X₅ comprises a D, E, A, G, I, L, M, W, F, P, or V, X₆ comprises a C, S, T, Y, N, or Q, X₇ comprises a C, S, T, Y, N, Q, A, G, I, L, M, W, F, P, or V, X₈ comprises a D, E, C, S, T, Y, N, or Q, X₉ comprises an A, G, I, L, M, W, F, P, or V. In some embodiments, X₁ comprises a V or L, X₂ comprises an F or I, X₃ comprises a Y, X₄ comprises a Y or T, X₅ comprises a D or A, X₆ comprises an S, X₇ comprises a Q or A, X₈ comprises an E or S, and X₉ comprises a V or I. In some embodiments, the LCDR2 comprises the amino acid sequence VFYTDSAEV (SEQ ID NO:65). In some embodiments, the LCDR2 comprises the amino acid sequence LIYYASQSI (SEQ ID NO:66).

In some embodiments, the light chain complementarity determining region 3 (LCDR3) comprising 3-25 amino acids in length. In some embodiments, the LCDR3 is 5-15 amino acids in length. In some embodiments, the LCDR3 is 6-10 amino acids in length. In some embodiments the LCDR3 is 3 amino acids in length. In some embodiments the LCDR3 is 4 amino acids in length. In some embodiments the LCDR3 is 5 amino acids in length. In some embodiments the LCDR3 is 6 amino acids in length. In some embodiments the LCDR3 is 7 amino acids in length. In some embodiments the LCDR3 is 8 amino acids in length. In some embodiments the LCDR3 is 9 amino acids in length. In some embodiments the LCDR3 is 10 amino acids in length. In some embodiments the LCDR3 is 11 amino acids in length. In some embodiments the LCDR3 is 12 amino acids in length. In some embodiments the LCDR3 is 13 amino acids in length. In some embodiments the LCDR3 is 14 amino acids in length. In some embodiments the LCDR3 is 15 amino acids in length. In some embodiments the LCDR3 is 16 amino acids in length. In some embodiments the LCDR3 is 17 amino acids in length. In some embodiments the LCDR3 is 18 amino acids in length. In some embodiments the LCDR3 is 19 amino acids in length. In some embodiments the LCDR3 is 20 amino acids in length. In some embodiments the LCDR3 is 21 amino acids in length. In some embodiments the LCDR3 is 22 amino acids in length. In some embodiments the LCDR3 is 23 amino acids in length. In some embodiments the LCDR3 is 24 amino acids in length. In some embodiments the LCDR3 is 25 amino acids in length. In some embodiments the LCDR3 comprises the amino acid sequence X₁X₂X₃X₄X₅X₆X₇X₈X₉, wherein X₁ comprises a polar amino acid, wherein X₂ comprises a polar amino acid, wherein X₃ comprises a nonpolar amino acid, wherein X₄ comprises a basic amino acid, wherein X₅ comprises a polar amino acid, wherein X₆ comprises a nonpolar amino acid, wherein X₇ comprises a nonpolar amino acid, wherein X₈ comprises a polar amino acid, and wherein X₉ comprises a polar amino acid. In some embodiments, X₁ comprises a C, S, T, Y, N, or Q, X₂ comprises a C, S, T, Y, N, or Q, X₃ comprises an A, G, I, L, M, W, F, P, or V, X₄ comprises an H, K or R, X₅ comprises a C, S, T, Y, N, or Q, X₆ comprises an A, G, I, L, M, W, F, P, or V, X₇ comprises an A, G, I, L, M, W, F, P, or V, X₈ comprises a C, S, T, Y, N, or Q, and X₉ comprises a C, S, T, Y, N, or Q. In some embodiments, X₁ comprises a Q, X₂ comprises an N, X₃ comprises a G, X₄ comprises an H, X₅ comprises an S, X₆ comprises an F, X₇ comprises a P, X₈ comprises a Y, and X₉ comprises a T. In some embodiments, the LCDR3 comprises the amino acid sequence QNGHSFPYT (SEQ ID NO:67). In some embodiments, the LCDR3 comprises the amino acid sequence QQGHSFPYT (SEQ ID NO:68). In some embodiments, the LCDR3 comprises the amino acid sequence QSGHSFPYT (SEQ ID NO:69).

In certain embodiments, the anti-IL-5 binding protein comprises one or more (i.e. one, two, three, four, five, or all six) CDRs of felinized mouse clone 154 disclosed herein. In certain embodiments, the anti-IL-5 binding protein comprises one or more (i.e. one, two, three, four, five, or all six) CDRs of an affinity matured felinized antibody disclosed herein. In certain embodiments, the anti-IL-5 binding protein comprises CDRs from one or more of felinized mouse clone 154 and the affinity matured variants provided herein.

In certain embodiments, the binding proteins comprise a human or a humanized antibody. In certain embodiments, the binding proteins comprise a feline or a felinized antibody.

In certain embodiments, an amino acid residue is mutated into one that allows the properties of the amino acid side-chain to be conserved. Examples of the properties of amino acid side chains comprise: polar amino acids (C, S, T, Y, N, Q), nonpolar amino acids (A, G, I, L, M, W, F, P, V), basic amino acids (H, K, R), acidic amino acids (E, D), hydrophobic amino acids (A, I, L, M, F, P, W, Y, V), hydrophilic amino acids (R, D, N, C, E, Q, G, H, K, S, T), and amino acids comprising the following side chains: aliphatic side-chains (G, A, V, L, I, P); hydroxyl group-containing side-chains (S, T, Y); sulfur atom-containing side-chains (C, M); carboxylic acid- and amide-containing side-chains (D, N, E, Q); base-containing side-chains (R, K, H); and aromatic-containing side-chains (H, F, Y, W). The letters within parenthesis indicate the one-letter amino acid codes. Amino acid substitutions within each group are called conservative substitutions. It is well known that a polypeptide comprising a modified amino acid sequence in which one or more amino acid residues is deleted, added, and/or substituted can retain the original biological activity (Mark D. F. et al., Proc. Natl. Acad. Sci. U.S.A. 81:5662-5666 (1984); Zoller M. J. and Smith M., Nucleic Acids Res. 10: 6487-6500 (1982); Wang A. et al., Science 224: 1431-1433; Dalbadie-McFarland G. et al., Proc. Natl. Acad. Sci. U.S.A. 79: 6409-6413 (1982)). The number of mutated amino acids is not limited, but in general, the number falls within 40% of amino acids of each CDR, and preferably within 35%, and still more preferably within 30% (e.g., within 25%). The identity of amino acid sequences can be determined as described herein.

The invention provides recombinant antibodies designed or modified to minimize antigenicity in felines and humans. In certain embodiments, the antibodies are further modified to remove T cell epitopes.

As used herein, the term “feline” refers to any member of the Felidae family. Domestic cats, pure-bred and/or mongrel companion cats, and wild or feral cats are all felines.

As used herein the term “human framework” or “feline framework” refers to the amino acid sequence of the heavy chain and light chain of a feline antibody other than the hypervariable region residues defined herein as CDR residues. With regard to a humanized antibody, in certain embodiments, feline CDRs are identified in human antibody heavy and light chains variable domain sequences that closely match CDRs of IL-5-binding antibodies originating in other species. In certain embodiments, native human CDRs are replaced with the corresponding foreign CDRs (e.g., those from a rat or a mouse antibody) in both chains. With regard to a felinized antibody, in certain embodiments, feline CDRs are identified in feline antibody heavy and light chains variable domain sequences that closely match CDRs of IL-5-binding antibodies originating in other species. In certain embodiments, native feline CDRs are replaced with the corresponding foreign CDRs (e.g., those from a rat or a mouse antibody) in both chains. Optionally the heavy and/or light chains of the humanized or felinized antibody may contain some mutated or foreign non-CDR residues, e.g., framework amino acid residues that vary among germline antibody sequence or mutations that preserve the conformation of the foreign CDRs within the antibody.

Five major isotypes (IgA, IgG, IgM, IgD, IgE) and two forms of light chain (x and X) are present in dogs. In the dog, there are four subtypes for IgG, which are IgGA, IgGB, IgGC, and IgGD (Bergeron et al al, 2014, Comparative functional characterization of canine IgG subclasses. Veterinary Immunology and Immunopathology. 157:31-41). For the cat, there are three subtypes of IgG which are IgG1a, IgG1b, and IgG2 (Streitzel et al. 2014, In vitro functional characterization of feline IgGs. Vet Immunol Immunopathol 158, 214-223, doi.org/10.1016/j.vetimm.2014.01.012).

The invention provides caninized and felinized antibodies engineered to modulate one or more effector functions or circulation half-life. Hinge and constant domains of an antibody engage host receptors or complement protein to mediate effector functions and regulate antibody circulation. In certain embodiments, one or more effector function is enhanced. In certain embodiments, one or more effector function is reduced or eliminated. In certain embodiments, antibodies of the invention comprise modifications to modulate antibody-dependent cytotoxicity (ADCC) and/or complement-dependent cytotoxicity (CDC). A non-limiting example involves engineering of canine IgGB constant region residues Met242 and/or Leu243 (EU numbering) to reduce effector function (see, e.g., Lund et al., Human Fc gamma RI and Fc gamma RII interact with distinct but overlapping sites on human IgG. J Immunol., 1991, 147:2657-62). In certain embodiments, a IgGB constant region of the invention comprises M242A and L243A substitution. In certain embodiments, the second constant domain (CH2) and/or the third constant domain (CH3) comprises mutations and combinations of mutations from wild-type designed to modulate binding to FcRn (neonatal Fc) receptor. In canine constant regions, such mutations include, without limitation substitutions of Ala426, for example A426Y or A426H, substitutions of Thr286, for example T286L or T286Y, substitutions of Tyr436, for example Y436H, and combinations of such mutations including but not limited to A426Y+T286L, A426Y+Y436H, A426H+T286L, and A426H+T286Y. In certain embodiments a chimeric or caninized antibody of the invention comprises a substitution at amino acid Asn434, such as but not limited to N434H. In feline constant regions, such mutations include, without limitation substitutions of Ser428, including but not limited to S428Y or S428L, substitutions of Gln311, including but not limited to Q311V, substitutions of Leu309, including but not limited to L309V, substitutions of Thr286, including but not limited to T286E, substitutions of Glu380, including but not limited to E380T, and combinations of such mutations including but not limited to S428Y+Q311V, S428Y+L309V, S428Y+Q311V+T286E, S428Y+Q311V+E380T, and S428Y+L309V+E380T. In certain embodiments a chimeric or felinized antibody of the invention comprises a substitution at amino acid Ser428 and/or Ser434 including but not limited to S428L and/or S434H.

The term “antibody,” as used herein, includes antigen-binding fragments of full antibody molecules. The terms “antigen-binding portion” of an antibody, “antigen-binding fragment” of an antibody, and the like, as used herein, include any naturally occurring, enzymatically obtainable, synthetic, or genetically engineered polypeptide or glycoprotein that specifically binds an antigen to form a complex. As used herein, the term “specifically binds” or “binds specifically” means that an IL-5 binding protein of the invention reacts or associates more frequently, more rapidly, with greater duration and/or with greater affinity with IL-5 than it does with alternative antigens. For example, IL-5 binding protein binds to IL-5 with materially greater affinity (e.g., at least 2-fold or 5-fold or 10-fold or 20-fold or 50-fold or 100-fold or 500-fold or 1000-fold or 10,000-fold or greater) than it does to other proteins or peptides. In certain embodiments, the IL-5-binding proteins binds to IL-5 with an equilibrium dissociation constant KD for the epitope or target to which it binds of, e.g., 10-4 M or smaller, e.g., 10-5 M, 10-6 M, 10-7 M, 10-8 M, 10-9 M, 10-10 M, 10-11 M, or 10-12 M. It will be recognized by one of skill that an antibody that specifically binds to a target (e.g., IL-5) from one species may also specifically bind to orthologs of IL-5.

Antigen-binding fragments of an antibody may be derived, e.g., from full antibody molecules using any suitable standard techniques such as proteolytic digestion or recombinant genetic engineering techniques involving the manipulation and expression of DNA encoding antibody variable and optionally constant domains. Such DNA is known and/or is readily available from, e.g., commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

Non-limiting examples of antigen-binding fragments include: (i) Fab fragments; (ii) F(ab′)2 fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated complementarity determining region (CDR) such as a CDR3 peptide), or a constrained FR3-CDR3-FR4 peptide. Other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g. monovalent nanobodies, bivalent nanobodies, etc.), small modular immunopharmaceuticals (SMIPs), and shark variable IgNAR domains, are also encompassed within the expression “antigen-binding fragment,” as used herein.

In certain embodiments, an antigen-binding fragment of an antibody comprises at least one variable domain. The variable domain may be of any size or amino acid composition and will generally comprise at least one CDR which is adjacent to or in frame with one or more framework sequences. In antigen-binding fragments having a VH domain associated with a VL domain, the VH and VL domains may be situated relative to one another in any suitable arrangement. For example, the variable region may be dimeric and contain VH-VH, VH-VL or VL-VL dimers. Alternatively, the antigen-binding fragment of an antibody may contain a monomeric VH or VL domain.

In certain embodiments, an antigen-binding fragment of an antibody may contain at least one variable domain covalently linked to at least one constant domain. Non-limiting, exemplary configurations of variable and constant domains that may be found within an antigen-binding fragment of an antibody of the present invention include: (i) VH-CH1; (ii) VH-CH2; (iii) VH-CH3; (iv) VH-CH1-CH2; (V) VH-CH1-CH2-CH3; (vi) VH-CH2-CH3; (vii) VH-CL; (viii) VL-CH1; (ix) VL-CH2, (x) VL-CH3; (xi) VL-CH1-CH2; (xii) VL-CH1-CH2-CH3; (xiii) VL-CH2-CH3; and (xiv) VL-CL. In any configuration of variable and constant domains, including any of the exemplary configurations listed above, the variable and constant domains may be either directly linked to one another or may be linked by a full or partial hinge or linker region. A hinge region may consist of at least 2 (e.g., 5, 10, 15, 20, 40, 60 or more) amino acids which result in a flexible or semi-flexible linkage between adjacent variable and/or constant domains in a single polypeptide molecule. Moreover, an antigen-binding fragment of an antibody of the present invention may comprise a homo-dimer or hetero-dimer (or other multimer) of any of the variable and constant domain configurations listed above in non-covalent association with one another and/or with one or more monomeric VH or VL domain (e.g., by disulfide bond(s)).

The term “diabody (Db)” refers to a bivalent antibody fragment constructed by gene fusion (for example, P. Holliger et al., Proc. Natl. Acad. Sci. USA 90: 6444-6448 (1993), EP 404,097, WO 93/11161). In general, a diabody is a dimer of two polypeptide chains. In the each of the polypeptide chains, a light chain variable region (VL) and a heavy chain variable region (VH) in an identical chain are connected via a short linker, for example, a linker of about five residues, so that they cannot bind together. Because the linker between the two is too short, the VL and VH in the same polypeptide chain cannot form a single chain V region fragment, but instead form a dimer. Thus, a diabody has two antigen-binding domains. When the VL and VH regions against the two types of antigens (a and b) are combined to form VLa-VHb and VLb-VHa via a linker of about five residues, and then co-expressed, they are secreted as bispecific Dbs. The antibodies of the present invention may be such Dbs.

A single-chain antibody (also referred to as “scFv”) can be prepared by linking a heavy chain V region and a light chain V region of an antibody (for a review of scFv see Pluckthun “The Pharmacology of Monoclonal Antibodies” Vol. 113, eds. Rosenburg and Moore, Springer Verlag, N.Y., pp. 269-315 (1994)). Methods for preparing single-chain antibodies are known in the art (see, for example, U.S. Pat. Nos. 4,946,778; 5,260,203; 5,091,513; and 5,455,030). In such scFvs, the heavy chain V region and the light chain V region are linked together via a linker, preferably, a polypeptide linker (Huston, J. S. et al., Proc. Natl. Acad. Sci. U.S.A, 1988, 85, 5879-5883). The heavy chain V region and the light chain V region in a scFv may be derived from the same antibody, or from different antibodies. The peptide linker used to ligate the V regions may be any single-chain peptide consisting of 12 to 19 residues. A DNA encoding a scFv can be amplified by PCR using, as a template, either the entire DNA, or a partial DNA encoding a desired amino acid sequence, selected from a DNA encoding the heavy chain or the V region of the heavy chain of the above antibody, and a DNA encoding the light chain or the V region of the light chain of the above antibody; and using a primer pair that defines the two ends. Further amplification can be subsequently conducted using a combination of the DNA encoding the peptide linker portion, and the primer pair that defines both ends of the DNA to be ligated to the heavy and light chain respectively. After constructing DNAs encoding scFvs, conventional methods can be used to obtain expression vectors comprising these DNAs, and hosts transformed by these expression vectors. Furthermore, scFvs can be obtained according to conventional methods using the resulting hosts. These antibody fragments can be produced in hosts by obtaining genes that encode the antibody fragments and expressing these as outlined above. Antibodies bound to various types of molecules, such as polyethylene glycols (PEGs), may be used as modified antibodies. Methods for modifying antibodies are already established in the art. The term “antibody” in the present invention also encompasses the above-described antibodies.

The term “Kd” as used herein, refers to the dissociation constant of an antibody-antigen interaction. The dissociation constant, Kd, and the association constant, Ka, are quantitative measures of affinity. At equilibrium, free antigen (Ag) and free antibody (Ab) are in equilibrium with antigen-antibody complex (Ag-Ab), and the rate constants, ka and kd, quantitate the rates of the individual reactions. At equilibrium, ka [Ab][Ag]=kd [Ag-Ab]. The dissociation constant, Kd, is given by: Kd=kd/ka=[Ag][Ab]/[Ag-Ab]. Kd has units of concentration, most typically M, mM, nM, pM, etc. When comparing antibody affinities expressed as Kd, having greater affinity for IL-5 is indicated by a lower value. The association constant, Ka, is given by: Ka=ka/kd=[Ag-Ab]/[Ag][Ab]. Ka has units of inverse concentration, most typically M-1, mM-1, nM-1, pM-1, etc. As used herein, the term “avidity” refers to the strength of the antigen-antibody binding taking valency into account.

The antibodies obtained can be purified to homogeneity. The antibodies can be isolated and purified by a method routinely used to isolate and purify proteins. The antibodies can be isolated and purified by the combined use of one or more methods appropriately selected from column chromatography, filtration, ultrafiltration, salting out, dialysis, preparative polyacrylamide gel electrophoresis, and isoelectro-focusing, for example (Strategies for Protein Purification and Characterization: A Laboratory Course Manual, Daniel R. Marshak et al. eds., Cold Spring Harbor Laboratory Press (1996); Antibodies: A Laboratory Manual. Ed Harlow and David Lane, Cold Spring Harbor Laboratory, 1988). Such methods are not limited to those listed above. Chromatographic methods include affinity chromatography, ion exchange chromatography, hydrophobic chromatography, gel filtration, reverse-phase chromatography, and adsorption chromatography. These chromatographic methods can be practiced using liquid phase chromatography, such as HPLC and FPLC. Columns to be used in affinity chromatography include protein A columns and protein G columns. For example, protein A columns include Hyper D, POROS, and Sepharose F. F. (Pharmacia). Antibodies can also be purified by utilizing antigen binding, using carriers on which antigens have been immobilized.

As used herein, the term “therapeutic agent” refers to any agent or material that has a beneficial effect on the mammalian recipient. Thus, “therapeutic agent” embraces both therapeutic and prophylactic molecules having nucleic acid or protein components.

“Treating” as used herein refers to ameliorating at least one symptom of, curing and/or preventing the development of a given disease or condition.

The anti-IL-5 proteins described herein, including antibodies or fragments thereof, are useful for ameliorating, or reducing the symptoms of, or treating, or preventing, diseases and disorders associated with IL-5. The anti-IL-5 proteins or fragments, as well as combinations with other agent, are to be administered in a therapeutically effective amount to subjects in need of treatment of diseases and disorders associated with IL-5 in the form of a pharmaceutical composition as described herein.

In certain embodiments the method comprises ameliorating, or reducing the symptoms of, or treating, or preventing disease in a subject. In certain embodiments, the anti-IL-5 proteins, antibodies, or fragments thereof inhibit the association of IL-5 with IL-5 receptor and/or eosinophil receptors, for example administered alone or in conjunction with a second agent and are used to treat, ameliorate, reduce the symptoms of, or prevent lung diseases, cardiovascular diseases, cancers, infectious diseases, neurological diseases, allergic/inflammatory diseases, or metabolic diseases.

Nonlimiting examples of cardiovascular diseases the antibody compositions and methods are used for ameliorating, or reducing the symptoms of, or treating, or preventing include hypertension, cardiac toxicity of anti-cancer drugs, cardiac toxicity of anthracyclines, cardiac toxicity of quinolones, heart failure regardless of origin, ischemia, heart attack, stroke, atherosclerosis, cardiac fibrillation, hypertension, thrombosis and embolism.

Nonlimiting examples of infectious diseases the antibody compositions and methods are used for ameliorating, or reducing the symptoms of, or treating, or preventing include AIDS, alveolar hydatid disease (AHD, echinococcosis), amebiasis (Entamoeba histolytica infection), Angiostrongylus infection, anisakiasis, anthrax, babesiosis (Babesia infection), Balantidium infection (balantidiasis), Baylisascaris infection (raccoon roundworm), bilharzia (schistosomiasis), Blastocystis hominis infection (blastomycosis), boreliosis, botulism, Brainerd diarrhea, brucellosis, bovine spongiform encephalopathy (BSE), candidiasis, capillariasis (Capillaria infection), chronic fatigue syndrome (CFS), Chagas disease (American trypanosomiasis), chickenpox (Varicella-Zoster virus), Chlamydia pneumoniae infection, cholera, Creutzfeldt-Jakob disease (CJD), clonorchiasis (Clonorchis infection), cutaneous larva migrans (CLM) (hookworm infection), coccidioidomycosis, conjunctivitis, Coxsackievirus A16 (hand, foot and mouth disease), cryptococcosis, Cryptosporidium infection (cryptosporidiosis), Culex mosquito (West Nile virus vector), cyclosporiasis (Cyclospora infection), cysticercosis (neurocysticercosis), Cytomegalovirus infection, Dengue/Dengue fever, Dipylidium infection (dog and cat flea tapeworm), Ebola virus hemorrhagic fever, encephalitis, Entamoeba coli infection, Entamoeba dispar infection, Entamoeba hartmanni infection, Entamoeba histolytica infection (amebiasis), Entamoeba polecki infection, enterobiasis (pinworm infection), enterovirus infection (non-polio), Epstein-Barr virus infection, Escherichia coli infection, foodborne infection, foot and mouth disease, fungal dermatitis, gastroenteritis, group A streptococcal disease, group B streptococcal disease, Hansen's disease (leprosy), Hantavirus pulmonary syndrome, head lice infestation (pediculosis), Helicobacter pylori infection, hematologic disease, Hendra virus infection, hepatitis (HCV, HBV), herpes zoster (shingles), HIV Infection, human ehrlichiosis, human parainfluenza virus infection, influenza, isosporiasis (Isospora infection), Lassa fever, leishmaniasis, Kala-azar (Kala-azar, Leishmania Infection), lice (body lice, head lice, pubic lice), Lyme disease, malaria, Marburg hemorrhagic fever, measles, meningitis, mosquito-borne diseases, Mycobacterium avium complex (MAC) infection, Naegleria infection, nosocomial infections, nonpathogenic intestinal ameobae infection, onchocerciasis (river blindness), opisthorciasis (Opisthorcis infection), parvovirus infection, plague, Pneumocystis carinii pneumonia (PCP), polio, Q fever, rabies, respiratory syncytial virus (RSV) Infection, rheumatic fever, Rift Valley fever, river blindness (onchocerciasis), rotavirus infection, roundworm infection, salmonellosis, Salmonella enteritidis, scabies, shigellosis, shingles, sleeping sickness, smallpox, streptococcal Infection, tapeworm infection (Taenia infection), tetanus, toxic shock syndrome, tuberculosis, ulcers (peptic ulcer disease), valley fever, Vibrio parahaemolyticus infection, Vibrio vulnificus infection, viral hemorrhagic fever, warts, waterborne infectious diseases, West Nile virus infection (West Nile encephalitis), whooping cough, yellow fever.

Nonlimiting examples of allergic/inflammatory conditions the antibody compositions and methods are used for ameliorating, or reducing the symptoms of, or treating, or preventing include, asthma, bronchial asthma, rheumatoid arthritis, inflammatory Bowel disease, type II diabetes, diabetes mellitus and deafness (DAD), Ballinger-Wallace syndrome, inflammatory diseases, rheumatic fever, pulmonary arterial hypertension, innate immune responses, cardiopulmonary diseases such as: chronic obstructive pulmonary disease, pulmonary embolism, pericarditis, coarctation of aorta, tetralogy of Fallot, aortic stenosis, mitral stenosis, aortic regurgitation, mitral regurgitation, pneumoconiosis, bronchiectasis, cardiomyopathies, and endothelial nitroglycerin tolerance.

Nonlimiting examples of lung diseases the antibody compositions and methods are used for ameliorating, or reducing the symptoms of, or treating, or preventing include, acute pneumonia, pulmonary fibrosis, interstitial pneumonia, pulmonary hypertension, chronic obstructive pulmonary disease (COPD), chronic bronchitis, pulmonary emphysema, asthma, refractory asthma, systemic inflammatory response syndrome (SIRS), lung injury acute (ALI), acute respiratory distress syndrome (ARDS), sarcoidosis, chronic idiopathic pulmonary thromboembolism, diffuse panbronchiolitis, cystic fibrosis, allergic alveolitis, lung cancer, obesity hypoventilation syndrome, alveolar hypoventilation syndrome and chronic transplant rejection pulmonary. Particularly important diseases are pulmonary fibrosis, interstitial pneumonia, pulmonary hypertension, asthma, COPD and SIRS.

Nonlimiting examples of cancers the antibody compositions and methods are used for ameliorating, or reducing the symptoms of, or treating, or preventing include cancers of the bladder, blood, bone, bone marrow, brain, breast, colon, esophagus, gastrointestinal tract, gum, head, kidney, liver, lung, nasopharynx, neck, ovary, prostate, skin, stomach, testis, tongue, or uterus, or malignant neoplasm, carcinoma, undifferentiated; giant and spindle cell carcinoma; small cell carcinoma; papillary carcinoma; squamous cell carcinoma; lymphoepithelial carcinoma; basal cell carcinoma; pilomatrix carcinoma; transitional cell carcinoma; papillary transitional cell carcinoma; adenocarcinoma; gastrinoma, malignant; cholangiocarcinoma; hepatocellular carcinoma; combined hepatocellular carcinoma and cholangiocarcinoma; trabecular adenocarcinoma; adenoid cystic carcinoma; adenocarcinoma in adenomatous polyp; adenocarcinoma, familial polyposis coli; solid carcinoma; carcinoid tumor, malignant; branchiolo-alveolar adenocarcinoma; papillary adenocarcinoma; chromophobe carcinoma; acidophil carcinoma; oxyphilic adenocarcinoma; basophil carcinoma; clear cell adenocarcinoma; granular cell carcinoma; follicular adenocarcinoma; papillary and follicular adenocarcinoma; nonencapsulating sclerosing carcinoma; adrenal cortical carcinoma; endometroid carcinoma; skin appendage carcinoma; apocrine adenocarcinoma; sebaceous adenocarcinoma; ceruminous; adenocarcinoma; mucoepidermoid carcinoma; cystadenocarcinoma; papillary cystadenocarcinoma; papillary serous cystadenocarcinoma; mucinous cystadenocarcinoma; mucinous adenocarcinoma; signet ring cell carcinoma; infiltrating duct carcinoma; medullary carcinoma; lobular carcinoma; inflammatory carcinoma; Paget's disease, mammary; acinar cell carcinoma; adenosquamous carcinoma; adenocarcinoma w/squamous metaplasia; thymoma, malignant; ovarian stromal tumor, malignant; thecoma, malignant; granulosa cell tumor, malignant; and roblastoma, malignant; Sertoli cell carcinoma; Leydig cell tumor, malignant; lipid cell tumor, malignant; paraganglioma, malignant; extra-mammary paraganglioma, malignant; pheochromocytoma; glomangiosarcoma; malignant melanoma; amelanotic melanoma; superficial spreading melanoma; malignant melanoma in giant pigmented nevus; epithelioid cell melanoma; blue nevus, malignant; sarcoma; fibrosarcoma; fibrous histiocytoma, malignant; myxosarcoma; liposarcoma; leiomyosarcoma; rhabdomyosarcoma; embryonal rhabdomyosarcoma; alveolar rhabdomyosarcoma; stromal sarcoma; mixed tumor, malignant; mullerian mixed tumor; nephroblastoma; hepatoblastoma; carcinosarcoma; mesenchymoma, malignant; brenner tumor, malignant; phyllodes tumor, malignant; synovial sarcoma; mesothelioma, malignant; dysgerminoma; embryonal carcinoma; teratoma, malignant; struma ovarii, malignant; choriocarcinoma; mesonephroma, malignant; hemangiosarcoma; hemangioendothelioma, malignant; kaposi's sarcoma; hemangiopericytoma, malignant; lymphangiosarcoma; osteosarcoma; juxtacortical osteosarcoma; chondrosarcoma; chondroblastoma, malignant; mesenchymal chondrosarcoma; giant cell tumor of bone; Ewing's sarcoma; odontogenic tumor, malignant; ameloblastic odontosarcoma; ameloblastoma, malignant; ameloblastic fibrosarcoma; pinealoma, malignant; chordoma; glioma, malignant; ependymoma; astrocytoma; protoplasmic astrocytoma; fibrillary astrocytoma; astroblastoma; glioblastoma; oligodendroglioma; oligodendroblastoma; primitive neuroectodermal; cerebellar sarcoma; ganglioneuroblastoma; neuroblastoma; retinoblastoma; olfactory neurogenic tumor; meningioma, malignant; neurofibrosarcoma; neurilemmoma, malignant; granular cell tumor, malignant; malignant lymphoma; Hodgkin's disease; Hodgkin's lymphoma; paragranuloma; malignant lymphoma, small lymphocytic; malignant lymphoma, large cell, diffuse; malignant lymphoma, follicular; mycosis fungoides; other specified non-Hodgkin's lymphomas; malignant histiocytosis; multiple myeloma; mast cell sarcoma; immunoproliferative small intestinal disease; leukemia; lymphoid leukemia; plasma cell leukemia; erythroleukemia; lymphosarcoma cell leukemia; myeloid leukemia; basophilic leukemia; eosinophilic leukemia; monocytic leukemia; mast cell leukemia; megakaryoblastic leukemia; myeloid sarcoma; and hairy cell leukemia.

Nonlimiting examples of neurological diseases the antibody compositions and methods are used for ameliorating, or reducing the symptoms of, or treating, or preventing include Alzheimer's disease, Parkinson's disease, Huntington's disease, Pick's disease, Kuf's disease, Lewy body disease, neurofibrillary tangles, Rosenthal fibers, Mallory's hyaline, senile dementia, myasthenia gravis, Gilles de la Tourette's syndrome, multiple sclerosis (MS), amyotrophic lateral sclerosis (ALS), progressive supranuclear palsy (PSP), epilepsy, Creutzfeldt-Jakob disease, deafness-dytonia syndrome, Leigh syndrome, Leber hereditary optic neuropathy (LHON), parkinsonism, dystonia, motor neuron disease, neuropathy-ataxia and retinitis pimentosa (NARP), maternal inherited Leigh syndrome (MILS), Friedreich ataxia, hereditary spastic paraplegia, Mohr-Tranebjaerg syndrome, Wilson disease, sporatic Alzheimer's disease, sporadic amyotrophic lateral sclerosis, sporadic Parkinson's disease, autonomic function disorders, hypertension, sleep disorders, neuropsychiatric disorders, depression, schizophrenia, schizoaffective disorder, korsakoffs psychosis, mania, anxiety disorders, phobic disorder, learning or memory disorders, amnesia or age-related memory loss, attention deficit disorder, dysthymic disorder, major depressive disorder, obsessive-compulsive disorder, psychoactive substance use disorders, panic disorder, bipolar affective disorder, severe bipolar affective (mood) disorder (BP-1), migraines, hyperactivity and movement disorders.

Nonlimiting examples of metabolic diseases the antibody compositions and methods are used for ameliorating, or reducing the symptoms of, or treating, or preventing include metabolic syndrome, diabetes (type 1 diabetes, type 2 diabetes, gestational diabetes, etc.), impaired glucose tolerance, obesity, diabetic neuropathy, diabetic retinopathy, diabetic nephropathy, dyslipidemia Diseases (hypertriglyceridemia, hypercholesterolemia, hypoHDLemia, postprandial hyperlipidemia, etc.), hypertension, hypertriglyceridemia, severe hypertriglyceridemia, hypercholesterolemia, familial, elevated cholesterol caused by a genetic condition, fatty liver disease, nonalcoholic fatty liver disease (NFLD), nonalcoholic steatohepatitis (NASH), dyslipidemia, mixed dyslipidemia, atherosclerosis, and coronary heart disease.

The anti-IL-5 proteins, antibodies or antibody fragments, are optionally administered in combination with one or more active agents including other analgesic agents. Such active agents include analgesic, anti-histamine, antipyretic, anti-inflammatory, antibiotic, antiviral, and anti-cytokine agents. Active agents include agonists, antagonists, and modulators of TNF-α, IL-2, IL-4, IL-6, IL-10, IL-12, IL-13, IL-18, IFN-α, IFN-γ, BAFF, CXCL13, IP-10, VEGF, EPO, EGF, HRG, Hepatocyte Growth Factor (HGF), Hepcidin, including antibodies reactive against any of the foregoing, and antibodies reactive against any of their receptors. Active agents also include, without limitation, 2-arylpropionic acids, aceclofenac, acemetacin, acetylsalicylic acid (Aspirin), alclofenac, alminoprofen, amoxiprin, ampyrone, arylalkanoic acids, azapropazone, benorylate/benorilate, benoxaprofen, bromfenac, carprofen, celecoxib, choline magnesium salicylate, clofezone, COX-2 inhibitors, dexibuprofen, dexketoprofen, diclofenac, diflunisal, droxicam, ethenzamide, etodolac, etoricoxib, faislamine, fenamic acids, fenbufen, fenoprofen, flufenamic acid, flunoxaprofen, flurbiprofen, ibuprofen, ibuproxam, indometacin, indoprofen, kebuzone, ketoprofen, ketorolac, lomoxicam, loxoprofen, lumiracoxib, magnesium salicylate, meclofenamic acid, mefenamic acid, meloxicam, metamizole, methyl salicylate, mofebutazone, nabumetone, naproxen, n-arylanthranilic acids, nerve growth factor (NGF), oxametacin, oxaprozin, oxicams, oxyphenbutazone, parecoxib, phenazone, phenylbutazone, phenylbutazone, piroxicam, pirprofen, profens, proglumetacin, pyrazolidine derivatives, rofecoxib, salicyl salicylate, salicylamide, salicylates, sulfinpyrazone, sulindac, suprofen, tenoxicam, tiaprofenic acid, tolfenamic acid, tolmetin, and valdecoxib.

An anti-histamine can be any compound that opposes the action of histamine or its release from cells (e.g., mast cells). Anti-histamines include but are not limited to acrivastine, astemizole, azatadine, azelastine, betatastine, brompheniramine, buclizine, cetirizine, cetirizine analogues, chlorpheniramine, clemastine, CS 560, cyproheptadine, desloratadine, dexchlorpheniramine, ebastine, epinastine, fexofenadine, HSR 609, hydroxyzine, levocabastine, loratidine, methscopolamine, mizolastine, norastemizole, phenindamine, promethazine, pyrilamine, terfenadine, and tranilast.

Antibiotics include but are not limited to amikacin, aminoglycosides, amoxicillin, ampicillin, ansamycins, arsphenamine, azithromycin, azlocillin, aztreonam, bacitracin, carbacephem, carbapenems, carbenicillin, cefaclor, cefadroxil, cefalexin, cefalothin, cefalotin, cefamandole, cefazolin, cefdinir, cefditoren, cefepime, cefixime, cefoperazone, cefotaxime, cefoxitin, cefpodoxime, cefprozil, ceftazidime, ceftibuten, ceftizoxime, ceftobiprole, ceftriaxone, cefuroxime, cephalosporins, chloramphenicol, cilastatin, ciprofloxacin, clarithromycin, clindamycin, cloxacillin, colistin, co-trimoxazole, dalfopristin, demeclocycline, dicloxacillin, dirithromycin, doripenem, doxycycline, enoxacin, ertapenem, erythromycin, ethambutol, flucloxacillin, fosfomycin, furazolidone, fusidic acid, gatifloxacin, geldanamycin, gentamicin, glycopeptides, herbimycin, imipenem, isoniazid, kanamycin, levofloxacin, lincomycin, linezolid, lomefloxacin, loracarbef, macrolides, mafenide, meropenem, meticillin, metronidazole, mezlocillin, minocycline, monobactams, moxifloxacin, mupirocin, nafcillin, neomycin, netilmicin, nitrofurantoin, norfloxacin, ofloxacin, oxacillin, oxytetracycline, paromomycin, penicillin, penicillins, piperacillin, platensimycin, polymyxin B, polypeptides, prontosil, pyrazinamide, quinolones, quinupristin, rifampicin, rifampin, roxithromycin, spectinomycin, streptomycin, sulfacetamide, sulfamethizole, sulfanilimide, sulfasalazine, sulfisoxazole, sulfonamides, teicoplanin, telithromycin, tetracycline, tetracyclines, ticarcillin, tinidazole, tobramycin, trimethoprim, trimethoprim-sulfamethoxazole, troleandomycin, trovafloxacin, and vancomycin.

Active agents also include aldosterone, beclometasone, betamethasone, corticosteroids, cortisol, cortisone acetate, deoxycorticosterone acetate, dexamethasone, fludrocortisone acetate, glucocorticoids, hydrocortisone, methylprednisolone, prednisolone, prednisone, steroids, and triamcinolone. Any suitable combination of these active agents is also contemplated.

### Formulations and Methods of Administration

For in vivo use, a therapeutic agent as described herein is generally incorporated into a pharmaceutical composition prior to administration. Within such compositions, one or more therapeutic compounds as described herein are present as active ingredient(s) (i.e., are present at levels sufficient to provide a statistically significant effect on the symptoms of cystic fibrosis, as measured using a representative assay). A pharmaceutical composition comprises one or more such compounds in combination with any pharmaceutically acceptable carrier(s) known to those skilled in the art to be suitable for the particular mode of administration. In addition, other pharmaceutically active ingredients (including other therapeutic agents) may, but need not, be present within the composition.

The antibodies of the present invention can be formulated according to standard methods (see, for example, Remington's Pharmaceutical Science, latest edition, Mark Publishing Company, Easton, U.S.A), and may comprise pharmaceutically acceptable carriers and/or additives. The present invention relates to compositions (including reagents and pharmaceuticals) comprising the antibodies of the invention, and pharmaceutically acceptable carriers and/or additives. Exemplary carriers include surfactants (for example, PEG and Tween), excipients, antioxidants (for example, ascorbic acid), coloring agents, flavoring agents, preservatives, stabilizers, buffering agents (for example, phosphoric acid, citric acid, and other organic acids), chelating agents (for example, EDTA), suspending agents, isotonizing agents, binders, disintegrators, lubricants, fluidity promoters, and corrigents. However, the carriers that may be employed in the present invention are not limited to this list. In fact, other commonly used carriers can be appropriately employed: light anhydrous silicic acid, lactose, crystalline cellulose, mannitol, starch, carmelose calcium, carmelose sodium, hydroxypropylcellulose, hydroxypropylmethyl cellulose, polyvinylacetaldiethylaminoacetate, polyvinylpyrrolidone, gelatin, medium chain fatty acid triglyceride, polyoxyethylene hydrogenated castor oil 60, sucrose, carboxymethylcellulose, corn starch, inorganic salt, and so on. The composition may also comprise other low-molecular-weight polypeptides, proteins such as serum albumin, gelatin, and immunoglobulin, and amino acids such as glycine, glutamine, asparagine, arginine, and lysine. When the composition is prepared as an aqueous solution for injection, it can comprise an isotonic solution comprising, for example, physiological saline, dextrose, and other adjuvants, including, for example, D-sorbitol, D-mannose, D-mannitol, and sodium chloride, which can also contain an appropriate solubilizing agent, for example, alcohol (for example, ethanol), polyalcohol (for example, propylene glycol and PEG), and non-ionic detergent (polysorbate 80 and HCO-50).

If necessary, antibodies of the present invention may be encapsulated in microcapsules (microcapsules made of hydroxycellulose, gelatin, polymethylmethacrylate, and the like), and made into components of colloidal drug delivery systems (liposomes, albumin microspheres, microemulsions, nano-particles, and nano-capsules) (for example, see “Remington's Pharmaceutical Science 16th edition”, Oslo Ed. (1980)). Moreover, methods for making sustained-release drugs are known, and these can be applied for the antibodies of the present invention (Langer et al., J. Biomed. Mater. Res. 15: 167-277 (1981); Langer, Chem. Tech. 12: 98-105 (1982); U.S. Pat. No. 3,773,919; EP Patent Application No. 58,481; Sidman et al., Biopolymers 22: 547-556 (1983); EP: 133,988).

The term “therapeutically effective amount,” in reference to treating a disease state/condition, refers to an amount of a compound either alone or as contained in a pharmaceutical composition that is capable of having any detectable, positive effect on any symptom, aspect, or characteristics of a disease state/condition when administered as a single dose or in multiple doses. Such effect need not be absolute to be beneficial.

The terms “treat,” “treating” and “treatment” as used herein include administering a compound prior to the onset of clinical symptoms of a disease state/condition so as to prevent any symptom, as well as administering a compound after the onset of clinical symptoms of a disease state/condition so as to reduce or eliminate any symptom, aspect or characteristic of the disease state/condition. Such treating need not be absolute to be useful.

In certain embodiments, the present therapeutic agent may be systemically administered, e.g., orally, in combination with a pharmaceutically acceptable vehicle such as an inert diluent or an assimilable edible carrier. They may be enclosed in hard or soft shell gelatin capsules, may be compressed into tablets, or may be incorporated directly with the food of the patient's diet. For oral therapeutic administration, the active compound may be combined with one or more excipients and used in the form of ingestible tablets, buccal tablets, troches, capsules, elixirs, suspensions, syrups, wafers, and the like. Such compositions and preparations should contain at least 0.1% of active compound. The percentage of the compositions and preparations may, of course, be varied and may conveniently be between about 2 to about 60% of the weight of a given unit dosage form. The amount of active compound in such therapeutically useful compositions is such that an effective dosage level will be obtained.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound may be incorporated into sustained-release preparations and devices.

The active compound may also be administered intravenously or intraperitoneally by infusion or injection. Solutions of the active compound or its salts may be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders comprising the active ingredient that are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the active compound in the required amount in the appropriate solvent with various of the other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

Useful dosages of the compounds of the present invention can be determined by comparing their in vitro activity, and in vivo activity in animal models. In certain embodiments, a useful dose is from about 0.1 mg/kg to about 5 mg/kg or from about 0.5 mg/kg to about 2 mg/kg. Methods for the extrapolation of effective dosages in humans and animals of different sizes are known to the art; for example, see U.S. Pat. No. 4,938,949.

The amount of the compound, or an active salt or derivative thereof, required for use in treatment will vary not only with the particular salt selected but also with the route of administration, the nature of the condition being treated and the age and condition of the patient and will be ultimately at the discretion of the attendant physician or clinician.

In general, however, a suitable dose will be in the range of from about 0.5 to about 100 mg/kg, e.g., from about 10 to about 75 mg/kg of body weight per day, such as 3 to about 50 mg per kilogram body weight of the recipient per day, preferably in the range of 6 to 90 mg/kg/day, most preferably in the range of 15 to 60 mg/kg/day.

The compound is conveniently administered in unit dosage form; for example, containing 5 to 1000 mg, conveniently 10 to 750 mg, most conveniently, 50 to 500 mg of active ingredient per unit dosage form.

Ideally, the active ingredient should be administered to achieve peak plasma concentrations of the active compound of from about 0.5 to about 75 μM, preferably, about 1 to 50 M, most preferably, about 2 to about 30 M. This may be achieved, for example, by the intravenous injection of a 0.05 to 5% solution of the active ingredient, optionally in saline, or orally administered as a bolus containing about 1-100 mg of the active ingredient. Desirable blood levels may be maintained by continuous infusion to provide about 0.01-5.0 mg/kg/hr or by intermittent infusions containing about 0.4-15 mg/kg of the active ingredient(s).

The desired dose may conveniently be presented in a single dose or as divided doses administered at appropriate intervals, for example, as two, three, four or more sub-doses per day. The sub-dose itself may be further divided, e.g., into a number of discrete loosely spaced administrations.

Exemplary IL-5 receptor α (IL-5Rα) constructs useful for screening, identifying, and evaluating anti-IL-5 antibodies that block receptor binding include the following:

i) Signal sequence-extracellular domain (ECD) of the feline IL-5
receptor α-V5 epitope-TEV protease site-human IgG1 Fc
(SEQ ID NO: 1)
METDTLLLWVLLLWVPGSTG
DSLSDKKSLLLPPTNFTIKVTGLSQVLLRWESNPDQEPVNIELGYHVRINAPQEEDYETRNT
ESKRVTVLHKGFSASVQTVPWNGQPLPPSRWVSAELKAPPGSPGTSIVNLTCTTNTTVNNHT
PLRPYQVSLHCSWLAGKDAPEDTQYFLYYRYGSWTEECQEYRRDTLNRNTACWFPKTFISSR
GSEALAVHANGSSKHAPIKPFDQLFALHAIDRINPPVNVTAEIEGTCLSIQWEKPVSAFPIH
CFEYEVKIYNTRKKYFQTEKLMTNAFISIIDDISKYSVQVRATVSSMCROMGFWSEWSQPIY
VG
GKPIPNPLLGLDST
ENLYFQG
EPKSCDKTHTCPPCPAPELLGGPSVFLFPPKPKDTLMISRTPEVTCVVVDVSHEDPEVKFNW
YVDGVEVHNAKTKPREEQYNSTYRVVSVLTVLHQDWLNGKEYKCKVSNKALPAPIEKTISKA
KGQPREPQVYTLPPSRDELTKNQVSLTCLVKGFYPSDIAVEWESNGQPENNYKTTPPVLDSD
GSFFLYSKLTVDKSRWQQGNVFSCSVMHEALHNHYTQKSLSLSPG
ii) Signal sequence-extracellular domain (ECD) of the feline IL-5
receptor α-2X (Gly-Gly-Gly-Ser) (SEQ ID NO: 89)-human IgG1-AviTag
(SEQ ID NO: 2)
MGWSCIILFLVATATGVHS
DSLSDKKSLLLPPTNFTIKVTGLSQVLLRWESNPDQEPVNIELGYHVRINAPQEEDYETRNT
ESKRVTVLHKGFSASVQTVPWNGQPLPPSRWVSAELKAPPGSPGTSIVNLTCTTNTTVNNHT
PLRPYQVSLHCSWLAGKDAPEDTQYFLYYRYGSWTEECQEYRRDTLNRNTACWFPKTFISSR
GSEALAVHANGSSKHAPIKPFDQLFALHAIDRINPPVNVTAEIEGTCLSIQWEKPVSAFPIH
CFEYEVKIYNTRKKYFQTEKLMTNAFISIIDDISKYSVQVRATVSSMCRQMGFWSEWSQPIY
VG
GGGSGGGS
EPKSCDKTHTCPPCPAPELLGGPSVFLFPPKPKDTLMISRTPEVTCVVVDVSHEDPEVKFNW
YVDGVEVHNAKTKPREEQYNSTYRVVSVLTVLHQDWLNGKEYKCKVSNKALPAPIEKTISKA
KGQPREPQVYTLPPSRDELTKNQVSLTCLVKGFYPSDIAVEWESNGQPENNYKTTPPVLDSD
GSFFLYSKLTVDKSRWQQGNVFSCSVMHEALHNHYTQKSLSLSPG
GLNDIFEAQKIEWHE

Although the present invention and its advantages have been described in detail, it should be understood that various changes, substitutions and alterations can be made herein without departing from the spirit and scope of the invention as defined in the appended claims.

The present invention will be further illustrated in the following Examples which are given for illustration purposes only and are not intended to limit the invention in any way.

### EXAMPLES

### Example 1: Generation and Characterization of Murine Antibody 154 that Binds to Feline IL-5

Five Balb/C mice were immunized with feline IL-5 (NP_001009845) fused to mouse IgG2a Fc (BAC44883.1) using an AbCellera proprietary method. The titers were measured in a flow cytometry assay using feline IL-5-coated beads. Serum at different dilutions were incubated with feline IL-5 (Kingfisher Biotech; RP1152F) coated beads for 30 minutes at 37° C. Beads were washed and binding of serum antibodies to beads was detected using a fluorescently labelled anti-mouse IgG secondary antibody (Jackson ImmunoResearch). Fluorescence was measured using high throughput plate-based flow cytometry on an INTELLICYT® IQUE® Screener Plus. Lymph nodes, spleen and bone marrow were collected from the three mice with significant IL-5 titres. The cells from all tissues were isolated and enriched for plasma cells using flow cytometry. An enriched plasma cell suspension was injected into an AbCellera microfluidic screening device. A total of 590,000 single cells were screened in the microfluidic device. Single cells secreting feline IL-5-specific antibodies were identified and isolated using a bead-based assay. Beads coated with anti-mouse IgG antibody (Jackson ImmunoResearch) were flowed onto microfluidic screening devices and incubated with single antibody-secreting cells. The IgG secreted by plasma cells were captured on beads using the constant region. Binding to secreted IgG immobilized on beads was subsequently assessed using fluorescently labeled feline IL-5 antigen. Positive hits were identified using machine vision and recovered using automated robotics-based protocols.

Single cell polymerase chain reaction (PCR) and custom molecular biology protocols generated NGS sequencing libraries (MiSeq, Illumina) using automated workstations (Bravo, Agilent). Sequencing data were analyzed using a custom bioinformatics pipeline to yield paired heavy and light chain sequences for each recovered antibody-secreting cell. 576 binders were recovered from the screening device for sequencing and 456 high confidence sequences were obtained. 252 unique antibodies were identified and then annotated with the closest germline (V[D]J) genes and degree of somatic hypermutation. Antibodies were considered members of the same clonal family if they shared the same inferred heavy and light V and J genes and had the same CDR3 length. Ninety-three families were identified using this process. Forty-eight antibodies were selected for expression and purification based on diversity and lack of cysteine liabilities. The variable (V[D]J) region of each antibody chain was synthesized and inserted into a mammalian expression vector with either a mouse IgG2a constant domain or a kappa constant domain using a custom, automated high-throughput cloning pipeline. Heavy and light chain expression vectors were transiently transfected into HD-293F cells and the antibodies purified with protein A chromatography. The antibodies were formulated in phosphate buffered saline (PBS), pH 7.2.

Purified mAbs were quantified by UV absorption spectrophotometry at 280 nm.

The murine antibodies were characterized by evaluating the purity, thermal stability, binding to feline IL-5 and blocking feline IL-5 binding to IL-5 receptor α (IL-5Rα).

The purity of the expressed and purified mAbs was analyzed by denaturing capillary sodium dodecyl sulfate gel electrophoresis (SDS-PAGE).

The melting point (Tm) of antibodies was assessed by differential scanning fluorimetry (DSF) using the SYPRO™ Orange fluorescence probe (ThermoFisher Scientific; S6653). 6 μL of mAb solution at 350 g/mL in phosphate-buffered saline, pH 7.4 (PBS) was mixed with 6 μL of a 19× concentrated SYPRO™ Orange solution diluted in PBS. Thermal unfolding as assessed by a change in fluorescence was measured on a Bio-Rad C1000 Touch Thermal Cycler instrument (Bio-Rad Laboratories) using a CFX96 Real-Time System reader head (Bio-Rad Laboratories). The wavelengths for excitation and emission were 450-490 nm and 560-580 nm, respectively. The fluorescence signal was measured at a starting temperature of 25° C. and increased to 95° C. in 0.5° C./minute increments. Data was analyzed and melting curves integrated using the Bio-Rad CFX Maestro software (v1.1). The Tm was defined as the local minimum taken from the derivative of the melting curve.

To measure the binding affinity of the antibodies to feline IL-5, high-throughput SPR experiments were performed on a Carterra LSA instrument equipped with an HC-30M chip type (Carterra-bio) using a 384 ligand array format. The chip surface was first activated by flowing a freshly prepared 1:1:1 activation mix of 100 mM MES pH 5.5, 100 mM S-NHS, and 400 mM EDC for seven minutes. Antibodies diluted to 10 μg/mL in 10 mM sodium acetate, pH 4.25 buffer, 0.01% Tween were coupled to the chip surface for 10 min using the instrument 96 Multi Flow Channel printhead. The chip surface was quenched by flowing 1 M Ethanolamine for seven minutes, followed by two wash steps of 15 seconds each in 25 mM MES pH 5.5 buffer. Feline IL-5 (Kingfisher Biotech; RP1152F) was used at the following concentrations: 1000, 333.3, 111.1, 37.0, 12.3, 4.1, 1.4, 0.45, and 0.15 nM.

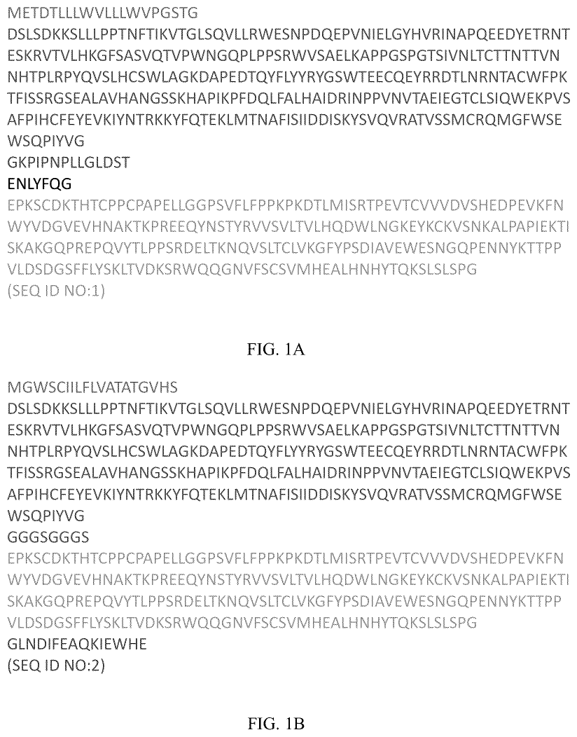

To determine the ability of the mouse anti-feline IL-5 antibodies to block the binding of a feline IL-5 to the feline IL-5 receptor α (IL-5Rα), an IL-5 receptor blocking assay was developed. For this assay, a fusion protein was generated containing the extracellular domain of feline IL-5Rα (XP_011278466.1) linked to the V5 epitope which is derived from a small epitope (Pk) found on the P and V proteins of the paramyxovirus of the simian virus 5 family linked to the TEV protease site linked to the human IgG1 Fc. The sequence of this construct is shown in FIG. 1A (SEQ ID NO:1). A signal sequence (METDTLLLWVLLLWVPGSTG; SEQ ID NO:70) was added to the N-terminus and the corresponding DNA construct was subcloned into pcDNA3.4 (Life Technologies). This construct was expressed with the Expi293 method (ThermoFisher Scientific) and the IL-5Rα-Fc construct was purified from the conditioned Expi293 medium using HiTrap protein A chromatography. The IL-5Rα-Fc construct was further purified with size exclusion chromatography using TOSOH TSKgel G3000Swxl column to isolate the dimeric form.

The receptor blocking assay was completed with a Biacore T200 instrument. The format for the Biacore assay was to conjugate the CM5 sensor chip with anti-human Fc antibody through amine coupling (Cytiva, cat #BR100839) to reach approximately 1,000 resonance units (RUs), bind IL-5 receptor alpha-Fc, and then flow over feline IL-5 (Kingfisher Biotech; Cat #RP1152F) or IL-5/Anti-IL-5 antibody mixtures (mixed at 1:1 ratio). The affinity of the feline IL-5 (Kingfisher Biotech; Cat #RP1152F-100) for the feline IL-5RA-Fc was 31 nM (ka=1.9E+5; kd=6.0E-3). The antibody and IL-5 were mixed at a 1:1 molar ratio and 12.5 nM, 25 nM and 50 nM concentrations of the antibody-IL-5 complexes were flowed over the receptor. The number of RUs observed when IL-5 was run alone was 60 RUs at 50 nM, 30 RUs at 25 nM and 15 RUs at 12.5 nM. The percent level of blocking was determined by the following equation: 100−[(mixture RUs/IL-5 RUs)×100].

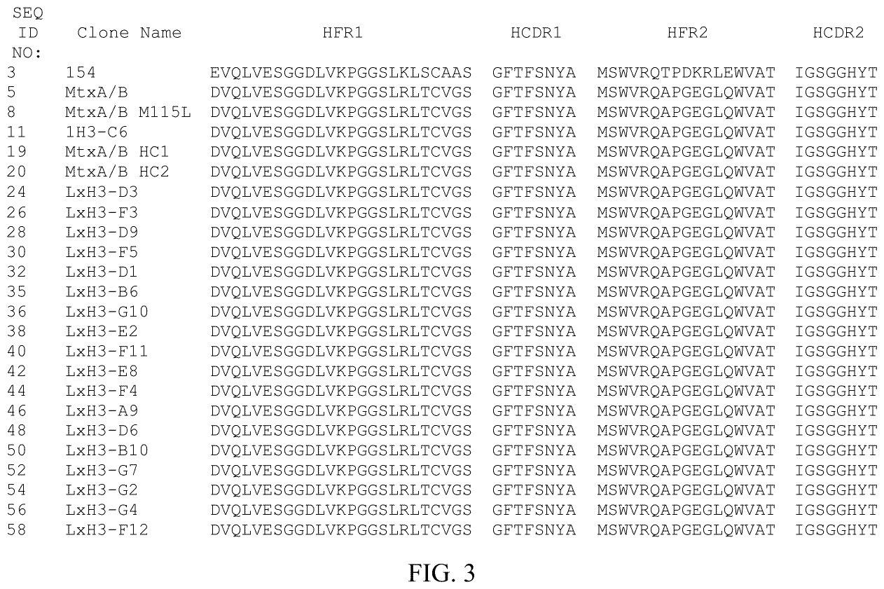

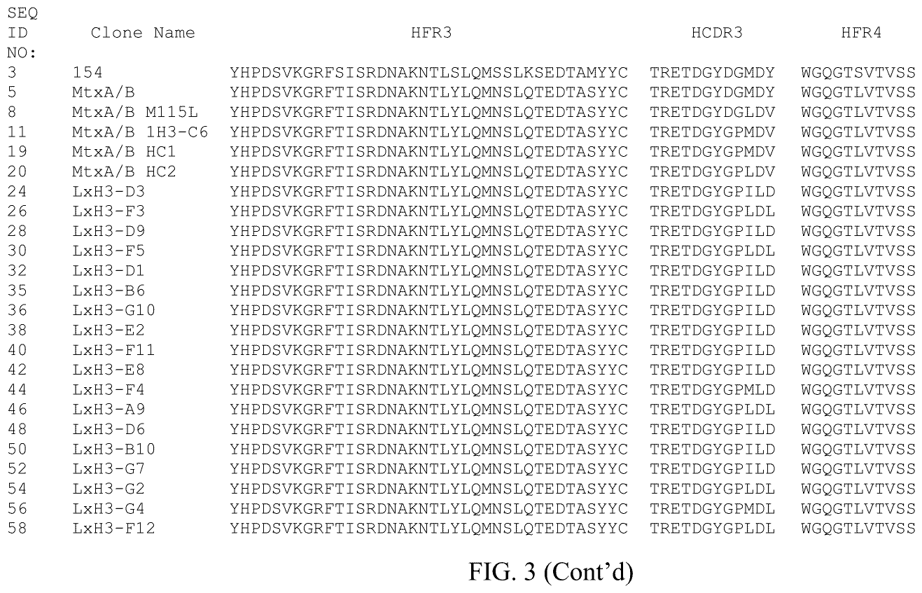

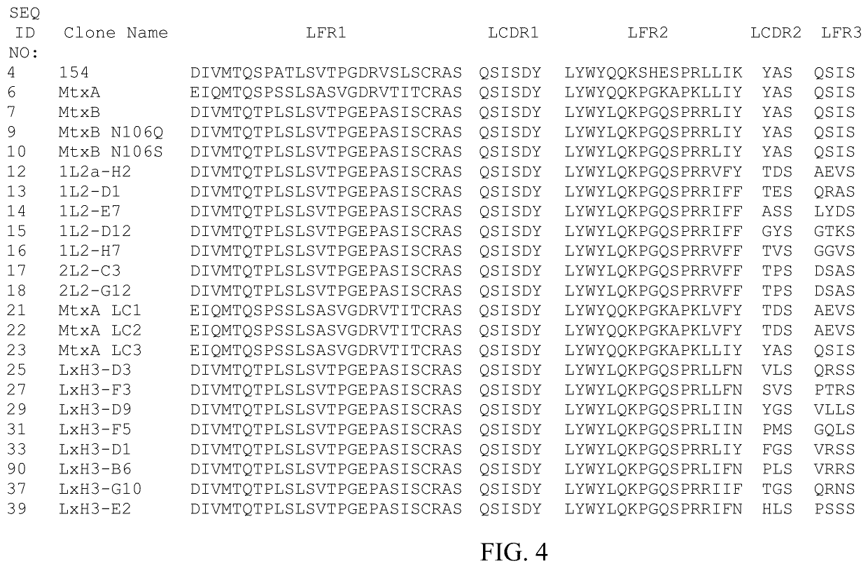

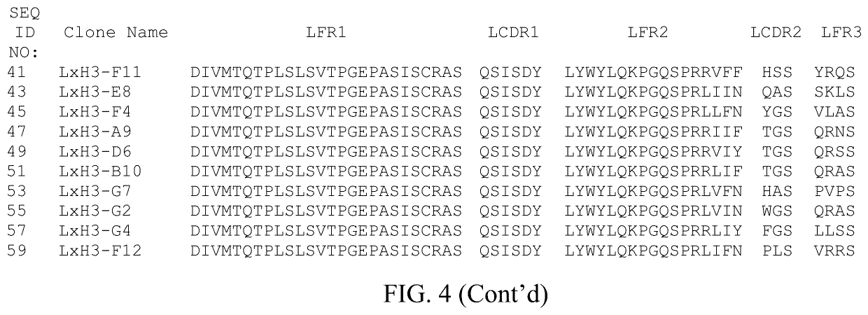

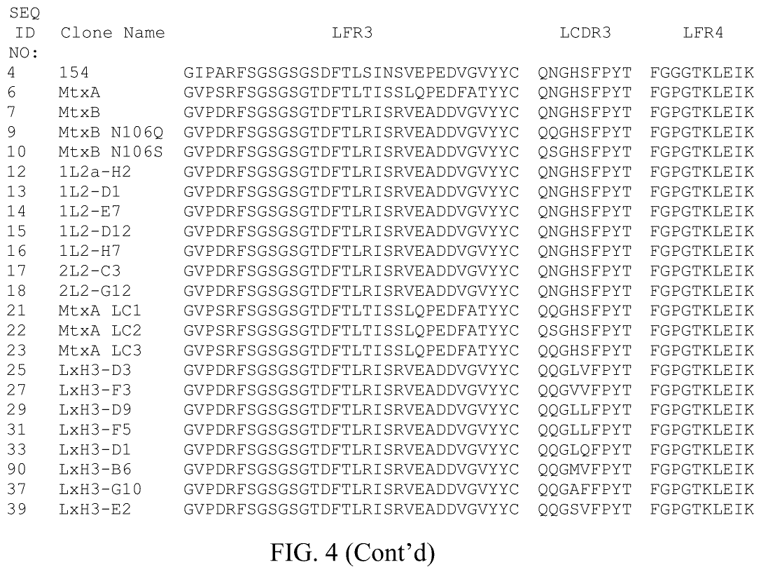

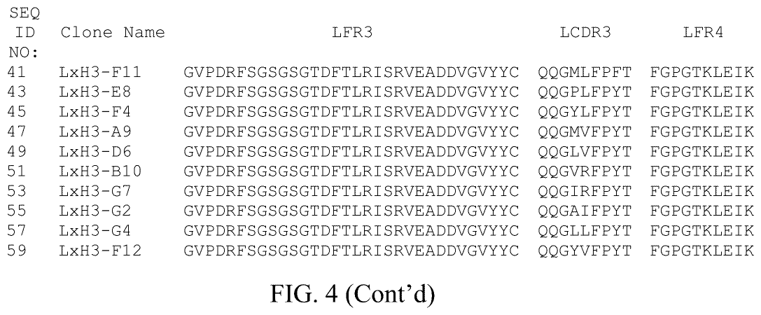

The mouse antibody clone selected for felinization was 154 and the variable domain sequences of the heavy chain (SEQ ID NO:3) and light chain (SEQ ID NO:4) variable domains are presented in FIG. 3 and FIG. 4 respectively. The CDR regions as defined by IMGT are indicated. For clone 154, Table 1 describes the percent purity of the antibody following purification, its thermal stability, the affinity to feline IL-5 and percentage of IL-5 receptor α blocking.

TABLE 1
Characterization of mouse antibodies

| | | | IL-5 affinity | % Receptor |
|---|---|---|---|---|
| | % purity | Tm (DSF) | (Carterra) | Blocking |
| Clone 154 | 99% | 61.5° C. | 1.41E−10 | 99% |

### Example 2: Felinization of Clone 154 and Characterization of Clones

For the felinization of clone 154, the frameworks of clone 154 were compared to the proprietary, expressed antibody database to find the feline frameworks with the highest identity. The proprietary expressed antibody database was generated by next-generation sequencing of feline PBMCs (peripheral blood mononuclear cells) and contains approximately 600,000 unique sequences for the VH (variable heavy), VL (variable lambda) and VK (variable kappa) domains. Twelve kappa feline frameworks were grafted onto the clone 154 kappa CDRs using the Kabat definition and two heavy feline frameworks were grafted onto the clone 154 heavy CDRs using the Kabat definition. The twelve feline kappa clones along with clone 154 were reformatted with the feline kappa constant domain (GenBank: ATI97438.1). The two feline heavy clones along with clone 154 were reformatted with the feline IgGla constant domains (GenBank: BAA32229.1). The twelve feline kappa clones and the two feline heavy chain constructs were subcloned into the pcDNA3.4 expression vector (ThermoFisher). The heavy and light chimeric clone 154 were also subcloned into a mammalian expression vector. All possible combinations of the heavy and light chain felinized clones along with the chimeric 154 clone were co-transfected into HEK 293 cells and the IgGs in the conditioned medium were purified with M abSelect SuRe protein A resin. The different purified felinized IgGs were referred to as Matrix clones (abbreviated as Mtx). The antibodies were buffered exchanged into 20 mM acetate, 136 mM NaCl, pH 5.5. By size exclusion chromatography (SEC) using Agilent AdvanceBio SEC 300A column none of the antibodies had more than 1% antibody aggregates.

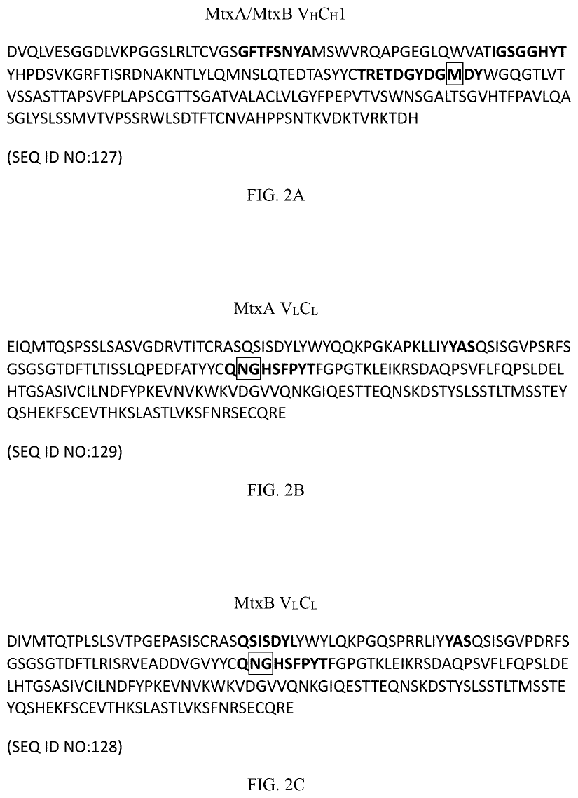

MtxA antibody and MtxB antibody had the highest affinity to feline IL-5 as determined by surface plasmon resonance (SPR) using a Biacore T200 instrument. Goat anti-feline IgG-Fc fragment specific (Jackson ImmunoResearch; 102-005-008) at 30 μg/ml in 10 mM acetate, pH 5.0 was immobilized onto CM5 sensor chip (Cytiva; 29104988) with NHS coupling at 10 μL/min for 420 seconds and then other sites blocked with ethanolamine at 10 μL/min for 420 seconds. The anti-feline IL-5 antibodies were captured at 2 μg/ml onto the anti-feline IgG CM5 chip for 60 sec at 10 μl/min. The feline IL-5 (Kingfisher Biotech; RP1152F) was run as the analyte at 5 dilutions (100, 50, 25, 12.5, 6.25 nM) onto the sensor chip at 30 μl/min for 120 sec and disassociated for 600 sec in PBS-P+(Cytiva; 28995084). The kinetics of the feline IL-5 binding to the antibodies is shown below in FIG. 2. In addition, an IL-5 receptor blocking assay was completed by capturing biotinylated receptors on a Series S CAP chip and Biotin CAPture reagent (Cytiva). For this assay, a fusion protein was generated containing the extracellular domain of feline IL-5Rα (XP_011278466.1) linked to a 2X-Gly-Gly-Gly-Ser (SEQ ID NO:89) linker-linked to the human IgG1 Fc linked to the AviTag sequence (FIG. 1B, SEQ ID NO:2) and subcloned into the pcDNA3.4 mammalian expression vector. This construct was expressed in CHO-S cells and purified with protein A chromatography followed by a polishing chromatography step with SEC. The purified recombinant protein was biotinylated in vitro using the BirA biotin ligase. Antibodies were titrated in running buffer (1×PBSP+, Cytiva) and pre-incubated with 50 nM IL-5 (Kingfisher) at multiple molar ratios. Binding was assessed by injecting these samples over the captured receptor for 180 s. The Rmax was used to calculate the inhibition percent by dividing the Rmax of the pre-mixed samples by an average of the IL-5-only Rmax samples that were collected throughout the assay. The ability of each antibody to block binding of feline IL5 was measured (Table 2).

TABLE 2
Characterization of felinized antibodies

| Clone | ka (1/Ms) | kd (1/s) | KD (M) | % blocking 1:1 |
|---|---|---|---|---|
| Chimeric 154 | 2.76E+05 | 3.56E−05 | 1.29E−10 | 98.2% |
| MtxA | 8.71E+04 | 1.49E−05 | 1.78E−10 | 92.5% |
| MtxB | 1.41E+05 | 5.48E−04 | 5.02E−09 | 74.2% |

The variable domain and first constant domain sequences of the MtxA and MtxB antibodies are shown in FIG. 2A-C(SEQ ID NO:127-129). Both antibodies comprise the same heavy chain variable domain (SEQ ID NO:5). The MtxA light chain variable domain (SEQ ID NO:6) and the MtxB light chain variable domain (SEQ ID NO:7) are each compatible with the common heavy chain variable domain. The individual frameworks and CDRs of MtxA derived light chain variable domains are interchangeable with the corresponding frameworks and CDRs of MtxB derived light chain variable domains.

### Example 3: Removing Potential Sequence Liabilities in LCDR3 and HCDR3

Asparagine deamidation for asparagine residues and oxidation at methionine residues can negatively impact the potency and the stability of an antibody (Xu et al., 2019. MABS, 11:239-264). These post-translational modifications can be particularly problematic when they reside in the CDRs. In MtxA and MtxB clones there is a potential deamidation site (NG) in LCDR3 (boxed in FIG. 2B (SEQ ID NO:129) and 2C (SEQ ID NO:128)) and a potential oxidation site (M) in HCDR3 (boxed in FIG. 2A (SEQ ID NO:127)). DNA constructs were synthesized (GeneArt) encoding the MtxB clone with substitutions to eliminate the potential sequence liabilities. For the NG site in LCDR3, the Asn (position 106) was substituted with either a Gln or a Ser and for the Met in HCDR3 (position 115), a Len was substituted. The DNA constructs with the light chain variants were fused to the feline kappa constant domain. These DNA constructs were subcloned into the pcDNA3.4 expression vector (ThermoFisher) and expressed with the ExpiCHO system (ThermoFisher). The antibodies were purified with mAbSelect SuRe chromatography (Cytiva) and buffer exchanged into 20 mM sodium acetate, 136 mM sodium chloride, pH 5.5. The variants were tested in an SPR experiment using a Biacore T200 instrument to measure their affinity to feline IL-5 (Kingfisher Biotech; RP1152F). Goat anti-feline IgG was immobilized onto a Series S CM5 sensor chip (Cytiva) and the original MtxB clone and the variants containing the amino acid substitutions were captured on the chip at 5 μg/ml. Feline IL-5 (100 nM, 50 nM, 25 nM, 12.5 nM, 6.25 nM and 0 nM) was flowed over with a contact time of 120 s, dissociation time of 600 s at a flow rate of 30 μl/min. The kinetic parameters of the mutated MatrixB antibodies are shown in Table 3.

TABLE 3
Characterization of MtxB mutants

| Clone | ka (1/Ms) | kd (1/s) | K_{D} (M) |
|---|---|---|---|
| MtxB | 9.0E+4 | 5.3E−4 | 5.9E−9 |
| N106Q (LCDR3) | 1.5E+5 | 8.6E−5 | 5.6E−10 |
| N106S (LCDR3) | 7.5E+4 | 2.7E−4 | 3.6E−9 |
| M115L (HCDR3) | 1.0E+5 | 1.5E−4 | 1.5E−9 |

### Example 4: Affinity Maturation of Felinized MtxB Clone

To improve the affinity of the MtxB clone to feline IL-5, an affinity maturation project was completed. An overview of the STEM (STage-Enhanced Maturation) platform used for the affinity maturation strategy is as follows. A high diversity of CDR sequences was evaluated by performing an iterative library construction and selection/screening process. In Stage A1, six separate CDR phage libraries were designed with maximal diversity, while constraining amino acid usage at each position to those predicted to be tolerated from bioinformatic and structural prediction analysis. These separate libraries were selected on antigen to create a pool of functional CDR mutants. Following screening, the CDR pools from the best performing libraries are PCR amplified and paired by overlap PCR to create a combined library in Stage B. This library was then selected under stringent conditions to obtain improved affinity candidates.

To generate the six separate libraries for each CDR, the light chain of MtxB (SEQ ID NO:7) was subcloned into a phagemid vector. For the heavy chain, the variable domain, CH1 domain and the partial hinge region (SEQ ID NO:5) were fused to a truncated pIII and subcloned into a phagemid vector. Primers were designed to introduce targeted mutations in each CDR separately and certain residues adjacent to the CDRs (Table 4).

TABLE 4
CDR regions targeted for mutation

| HCDR1 | HCDR2 | HCDR3 |
|---|---|---|
| VGSGFTFSNYAMS | ATIGSGGHYTYHP | TRETDGYDGMDY |
| (SEQ ID NO: 72) | (SEQ ID NO: 73) | (SEQ ID NO: 63) |
| LCDR1 | LCDR2 | LCDR3 |
| RASQSISDYLY | LIYYASQSI | QNGHSFPYT |
| (SEQ ID NO: 74) | (SEQ ID NO: 66) | (SEQ ID NO: 67) |

The mutagenic primers contained NNK codons for randomization of a targeted position to all 20 amino acids. The individual PCR fragments were paired for overlap PCR construction of the full gene library. The overlap PCR was performed in large scale, gel purified and subcloned into phagemid vectors. Each library was transformed into E. coli for growth and propagation of the Fab-phage. After overnight phage production, the phage was precipitated from the culture supernatant. The input amplified phage was incubated for 1 hour in a well of an Immulon 4 HBX high-binding plate that was coated with 10 μg/ml feline IL-5 (Kingfisher Biotech; Cat #RP1152F) and blocked with 1% bovine serum albumin in phosphate buffered saline (PBS), pH 7.4. The plate is then washed with PBS, pH 7.4, followed by acid elution. The output phage were then used to infect E. coli for amplification for the next round of panning. Washing was performed with either a low or a high stringency. The low stringency consisted of 3×3 min washes for each of the four rounds and high stringency conditions were 5×5 min washes for the first three rounds and 10×5 min wash for round 4. The number of phage input and output from each panning selection are measured by titering the phage. After the final round of selection, phage were plated onto a lawn of E. coli and 252 colonies from each selection strategy were grown in 96-well plates for growth and Fab production. To test for binding, Fab supernatant was pre-incubated with goat anti-feline IgG F(ab′)₂ to dimerize the Fab and then the complex was added to Immulon 4 HBX high-binding plate that was coated with 10 μg/ml feline IL-5 (Kingfisher Biotech; Cat #RP1152F) and blocked with 1% bovine serum albumin in phosphate buffered saline (PBS), pH 7.4. Donkey anti-goat antibody labeled with horse radish peroxidase was added to wells for detection of the Fab binding to feline IL-5. Clones that had higher binding than the original MtxB Fab, were sequenced and the unique clones were identified. Most of the positive clones were from the LCDR2 library and one positive clone from the HCDR3 library. A quantitative ELISA was used to normalize the concentrations of the positive Fab variants and then tested in feline IL-5 ELISA as described above. The sequences of the LCDR2 positive clones and the HCDR3 positive clone are shown in Table 5 along with the ELISA values in the feline IL-5 assay.

TABLE 5
Binding characteristics of MtxB mutants

| | | SEQ ID | Normalized fIL-5 ELISA value, |
|---|---|---|---|
| Clone | HCDR3 | NO: | 65 ng/ml Fab |
| 1H3-C6 | TRETDGYGPMDV | 71 | 0.967 |
| MtxB | TRETDGYDGMDY | 63 | 0.165 (Avg) |
| | | | Normalized fIL-5 ELISA value, |
| Clone | LCDR2 | | 65 ng/ml Fab |
| 1L2a-H2 | VFYTDSAEV | 65 | 0.559 |
| 1L2-D1 | IFFTESQRA | 75 | 0.492 |
| 1L2-E7 | IFFASSLYD | 76 | 0.366 |
| 1L2-D12 | IFFGYSGTK | 77 | 0.303 |
| 1L2-H7 | VFFTVSGGV | 78 | 0.283 |
| 2L2-C3 | VFFTPSDSA | 79 | 0.283 |
| 2L2-G12 | VFFTPSDSA | 79 | 0.251 |
| MtxB | LIYYASQSI | 66 | 0.165 (Avg) |

### Example 5: Combining Affinity Matured Variants to Eliminate Potential Sequence Liabilities

Antibodies containing the variants to remove the potential deamidation site and oxidation site were combined with the affinity maturation variants from stage A1 described above and tested for binding affinity to feline IL-5 and blocking feline IL-5 binding to feline IL-5RA. For this experiment, the frameworks of the variable domain are from MtxA (VH: SEQ ID NO:5; VL: SEQ ID NO:6) and the modified CDRs for each variant antibody chain (HCl, HC2, LC1, LC2, LC3) are shown below in Table 6.

TABLE 6
Engineered CDR variants

| | | | SEQ ID | | SEQ ID |
|---|---|---|---|---|---|
| Variant | Chain | HCDR3 (IMGT) | NO: | | NO: |
| HC1 | Heavy | TRETDGYGPMDV | 71 | | |
| HC2 | Heavy | TRETDGYGPLDV | 80 | | |
| Variant | Chain | LCDR2 (expanded) | | LCDR3 (IMGT) | |
| LC1 | Light | VFYTDSAEV | 65 | QQGHSFPYT | 68 |
| LC2 | Light | VFYTDSAEV | 65 | QSGHSFPYT | 69 |
| LC3 | Light | LIYYASQSI | 66 | QQGHSFPYT | 68 |

DNA constructs (GeneArt) were generated for the two heavy chain variants with the feline IgG1a constant domain along with the three light chain variants with the feline kappa constant domain were all subcloned into pcDNA 3.4 (ThermnoFisher Scientific). Each combination of the heavy and light chain variants was transfected into ExpiCHO cells to generate conditioned medium. The feline IgGs were purified using MabSelect SuRe chromatography and buffer exchanged into 20 mM sodium acetate, 136 mM sodium chloride, pH 5.5. The binding affinities of the different feline IgGs to feline IL-5 was determined using the same SPR method described in the removing potential sequence liabilities in LCDR3 and HCDR3 section and are shown below in Table 7.

TABLE 7
Binding to IL-5 of V_{H}V_{L} combinations

| Antibody | ka (1/Ms) | kd (1/s) | KD (M) |
|---|---|---|---|
| H2L3 | 1.85E+05 | 4.29E−06 | 2.20E−11 |
| H2L2 | 2.13E+05 | 9.11E−06 | 4.38E−11 |
| H1L1 | 9.41E+04 | 4.65E−06 | 5.00E−11 |
| H2L1 | 2.25E+05 | 1.20E−05 | 5.30E−11 |
| 154 chimera | 3.69E+05 | 3.38E−05 | 9.14E−11 |
| H1L3 | 1.96E+05 | 2.49E−05 | 1.26E−10 |
| MtxA | 2.42E+05 | 6.65E−05 | 2.73E−10 |

### Example 6: Additional Affinity Maturation of Felinized MtxB Clone

Another round of affinity maturation was completed using the STEM (STage-Enhanced Maturation) platform. For this round both Stage A1 and Stage B were completed as described above. The individual CDR libraries generated in Stage A1 were panned again to improve titers and improve the diversity of the CDR pools prior to Stage B library construction. For this panning, a higher concentration of 20 μg/ml was used to coat the 96 multi-wells to capture a higher diversity of clones. Additionally, transient heat treatment at 58° C. was used to eliminate any unstable clones.

For the Stage A1, 10 μg of CDR library were transformed into E. coli for growth and propagation of the Fab-phage. After overnight phage production, phage was precipitated from the culture supernatant and resuspended in blocking buffer (1% BSA/PBS). Transient heat treatment at 58° C. for 10 minutes was used to remove unstable clones from the library prior to selection. The input phage was then incubated for 1 hour in a 96 multi-well plate coated with 20 μg/ml of feline IL-5. The ELISA plate was then washed with PBS, pH 7.4, followed by acid elution. The output phage were used to infect E. coli for propagation for the next round of selection. The length of each wash was 5 minutes and three, five, five, and five washes were used for panning rounds one through four, respectively. An ELISA screening with feline IL-5 was performed on the output from the round four and the positive clones in the ELISA were sequenced. Good diversity was obtained from the LCDR2, LCDR3 and HCDR3 libraries.

Based on the screening results from the Stage A1 panning, libraries LCDR2, LCDR3 and HCDR3 were combined to create a Stage B library. Fragments containing the pre-selected CDR pools from these libraries were randomly paired by overlap PCR to create a new library incorporating mutations throughout all targeted CDRs. The phage propagated following round 4 of selection were used for PCR amplication of LCDR2, LCDR3 and HCDR3. The PCR-amplified fragment containing the light chain and the heavy chain were ligated into a phagemid vector and transformed into E. coli. The number of transformants was approximately 2E+10. Library plasmid DNA was purified using a midi-prep purification method. Ten pg of the library plasmid DNA was transformed into E. coli for growth and propagation of the Fab phage. After overnight phage production, phage was precipitated from the culture supernatant and resuspended in blocking buffer (1% BSA/PBS). The phage were treated at 58° C. for 10 minutes to remove unstable clones prior to the selections. The input phage was incubated for 30 minutes in a blocked ELISA plate coated with feline IL-5. The ELISA plate was washed with PBS, pH 7.4 followed by acid elution of the bound phage. The output phage was then used to infect E. coli for propagation for the next round of selection. For rounds 1, 2, 3 and 4, the ELISA plates were coated with 10 μg/ml, 8 μg/ml, 5 μg/ml and 1 μg/ml, respectively. The number of washes after each round were 3, 5, 10 and 10 for rounds 1, 2, 3 and 4, respectively. Ninety-three clones from the round 4 output were grown overnight for Fab production and tested for binding to feline IL-5 in an ELISA format. For this assay, supernatants from the overnight cultures were incubated in Immulon 4 HBX high-binding plate that was coated with 10 μg/ml feline IL-5 (Kingfisher Biotech; Cat #RP1152F) and blocked with 1% bovine serum albumin in phosphate buffered saline (PBS), pH 7.4. The controls added included the MtxB clone and clone 1H3-C6 which was the top affinity clone from the first stage A1 step. The Fab binding to the feline IL-5 was detected with anti-feline Fab′₂-HRP conjugate.

The results are shown in Table 8A which compares relative Fab binding affinity to feline IL-5 by Fabs disclosed herein. IL-5 binding was detected with anti-feline Fab′₂-HRP conjugate. Differences in CDR amino acids compared to the MtxB are underlined. The data in the last column are absorbance following addition of substrate to detect the HRP conjugate.

TABLE 8A

| Clone | LFR2 | LCDR2 | LCDR3 | HCDR3 | fIL5 Fab |
|---|---|---|---|---|---|
| Matrix B | WYLQKPGQSPRR | LIYYASQSI | QNGHSFPYT | TRETDGYDGMDY | 0.054 |
| 1H3-C6 | WYLQKPGQSPRR | LIYYASQSI | QNGHSFPYT | TRETDGYGPMDV | 0.100 |
| LxH3-D3 | WYLQKPGQSPRL | LFNVLSQRS | QQGLVFPYT | TRETDGYGPILD | 1.277 |
| LxH3-F3 | WYLQKPGQSPRL | LFNSVSPTR | QQGVVFPYT | TRETDGYGPLDL | 1.155 |
| LxH3-D9 | WYLQKPGQSPRL | IINYGSVLL | QQGLLFPYT | TRETDGYGPILD | 1.133 |
| LxH3-F5 | WYLQKPGQSPRL | IINPMSGQL | QQGLLFPYT | TRETDGYGPLDL | 1.042 |
| LxH3-D1 | WYLQKPGQSPRR | LIYFGSVRS | QQGLQFPYT | TRETDGYGPILD | 1.011 |
| LxH3-B6 | WYLQKPGQSPRL | IFNPLSVRR | QQGMVFPYT | TRETDGYGPILD | 0.972 |
| LxH3-G10 | WYLQKPGQSPRR | IIFTGSQRN | QQGAFFPYT | TRETDGYGPILD | 0.877 |
| LxH3-E2 | WYLQKPGQSPRR | IFNHLSPSS | QQGSVFPYT | TRETDGYGPILD | 0.809 |
| LxH3-F11 | WYLQKPGQSPRR | VFFHSSYRQ | QQGMLFPFT | TRETDGYGPILD | 0.782 |
| LxH3-E8 | WYLQKPGQSPRL | IINQASSKL | QQGPLFPYT | TRETDGYGPILD | 0.748 |
| LxH3-F4 | WYLQKPGQSPRL | LFNYGSVLA | QQGYLFPYT | TRETDGYGPMLD | 0.738 |
| LxH3-A9 | WYLQKPGQSPRR | IIFTGSQRN | QQGMVFPYT | TRETDGYGPLDL | 0.722 |
| LxH3-D6 | WYLQKPGQSPRR | VIYTGSQRS | QQGLVFPYT | TRETDGYGPILD | 0.708 |
| LxH3-B10 | WYLQKPGQSPRR | LIFTGSQRA | QQGVRFPYT | TRETDGYGPILD | 0.702 |
| LxH3-G7 | WYLQKPGQSPRL | VFNHASPVP | QQGIRFPYT | TRETDGYGPILD | 0.672 |
| LxH3-G2 | WYLQKPGQSPRL | VINWGSQRA | QQGAIFPYT | TRETDGYGPLDL | 0.653 |
| LxH3-G4 | WYLQKPGQSPRR | LIYFGSLLS | QQGLLFPYT | TRETDGYGPMDL | 0.545 |
| LxH3-F12 | WYLQKPGQSPRL | IFNPLSVRR | QQGYVFPYT | TRETDGYGPLDL | 0.513 |

TABLE 8B
SEQ ID NOs: for Table 8A

| CLONE | LFR2 | LCDR2 | LCDR3 | HCDR3 |
|---|---|---|---|---|
| Matrix B | SEQ ID NO: 91 | SEQ ID NO: 66 | SEQ ID NO: 67 | SEQ ID NO: 63 |
| 1H3-C6 | SEQ ID NO: 91 | SEQ ID NO: 66 | SEQ ID NO: 67 | SEQ ID NO: 71 |
| LxH3-D3 | SEQ ID NO: 92 | SEQ ID NO: 93 | SEQ ID NO: 109 | SEQ ID NO: 123 |
| LxH3-F3 | SEQ ID NO: 92 | SEQ ID NO: 94 | SEQ ID NO: 110 | SEQ ID NO: 125 |
| LxH3-D9 | SEQ ID NO: 92 | SEQ ID NO: 95 | SEQ ID NO: 111 | SEQ ID NO: 123 |
| LxH3-F5 | SEQ ID NO: 92 | SEQ ID NO: 96 | SEQ ID NO: 111 | SEQ ID NO: 125 |
| LxH3-D1 | SEQ ID NO: 91 | SEQ ID NO: 97 | SEQ ID NO: 112 | SEQ ID NO: 123 |
| LxH3-B6 | SEQ ID NO: 92 | SEQ ID NO: 98 | SEQ ID NO: 113 | SEQ ID NO: 123 |
| LxH3-G10 | SEQ ID NO: 91 | SEQ ID NO: 99 | SEQ ID NO: 114 | SEQ ID NO: 123 |
| LxH3-E2 | SEQ ID NO: 91 | SEQ ID NO: 100 | SEQ ID NO: 115 | SEQ ID NO: 123 |
| LxH3-F11 | SEQ ID NO: 91 | SEQ ID NO: 101 | SEQ ID NO: 116 | SEQ ID NO: 123 |
| LxH3-E8 | SEQ ID NO: 92 | SEQ ID NO: 102 | SEQ ID NO: 117 | SEQ ID NO: 123 |
| LxH3-F4 | SEQ ID NO: 92 | SEQ ID NO: 103 | SEQ ID NO: 118 | SEQ ID NO: 124 |
| LxH3-A9 | SEQ ID NO: 91 | SEQ ID NO: 99 | SEQ ID NO: 113 | SEQ ID NO: 125 |
| LxH3-D6 | SEQ ID NO: 91 | SEQ ID NO: 104 | SEQ ID NO: 109 | SEQ ID NO: 123 |
| LxH3-B10 | SEQ ID NO: 91 | SEQ ID NO: 105 | SEQ ID NO: 119 | SEQ ID NO: 123 |
| LxH3-G7 | SEQ ID NO: 92 | SEQ ID NO: 106 | SEQ ID NO: 120 | SEQ ID NO: 123 |
| LxH3-G2 | SEQ ID NO: 92 | SEQ ID NO: 107 | SEQ ID NO: 121 | SEQ ID NO: 125 |
| LxH3-G4 | SEQ ID NO: 91 | SEQ ID NO: 108 | SEQ ID NO: 111 | SEQ ID NO: 126 |
| LxH3-F12 | SEQ ID NO: 92 | SEQ ID NO: 98 | SEQ ID NO: 122 | SEQ ID NO: 125 |

Having thus described in detail preferred embodiments of the present invention, it is to be understood that the invention defined by the above paragraphs is not to be limited to particular details set forth in the above description as many apparent variations thereof are possible without departing from the spirit or scope of the present invention.

1. An antigen binding protein that specifically binds to interleukin-5 (IL-5), which comprises:
(a) a heavy chain complementarity determining region 1 (HCDR1) comprising X₁X₂X₃X₄X₅X₆X₇X₈, wherein X₁ comprises A, G, I, L, M, W, F, P, or V, X₂ comprises A, G, I, L, M, W, F, P, or V, X₃ comprises C, S, T, Y, N, or Q, X₄ comprises A, G, I, L, M, W, F, P, or V, X₅ comprises A, G, I, L, M, W, F, P, or V, X₆ comprises H, K, or R, X₇ comprises C, S, T, Y, N, or Q, and X₈ comprises C, S, T, Y, N, or Q;
(b) a heavy chain complementarity determining region 2 (HCDR2) comprising X₁X₂X₃X₄X₅X₆X₇X₈, wherein X₁ comprises A, G, I, L, M, W, F, P, or V, X₂ comprises A, G, I, L, M, W, F, P, or V, X₃ comprises C, S, T, Y, N, or Q, X₄ comprises A, G, I, L, M, W, F, P, or V, X₅ comprises A, G, I, L, M, W, F, P, or V, X₆ comprises H, K, or R, X₇ comprises C, S, T, Y, N, or Q, and X₈ comprises a C, S, T, Y, N, or Q;
(c) a heavy chain complementarity determining region 3 (HCDR3) comprising X₁X₂X₃X₄X₅X₆X₇X₈X₉X₁₀X₁₁X₁₂, wherein X₁ comprises C, S, T, Y, N, or Q, X₂ comprises H, K, or R, X₃ comprises an E or D, X₄ comprises C, S, T, Y, N, or Q, X₅ comprises E or D, X₆ comprises A, G, I, L, M, W, F, P, or V, X₇ comprises C, S, T, Y, N, or Q, X₈ comprises D, E, or G, X₉ comprises A, G, I, L, M, W, F, P, or V, X₁₀ comprises A, G, I, L, M, W, F, P, or V, X₁₁ comprises D, E, or L, and X₁₂ comprises C, L, S, T, V, Y, N, or Q;
(d) a light chain complementarity determining region 1 (LCDR1) comprising X₁X₂X₃X₄X₅X₆, wherein X₁ comprises C, S, T, Y, N, or Q, X₂ comprises C, S, T, Y, N, or Q, X₃ comprises A, G, I, L, M, W, F, P, or V, X₄ comprises C, S, T, Y, N, or Q, X₅ comprises E or D, and X₆ comprises C, S, T, Y, N, or Q;
(e) a light chain complementarity determining region 2 (LCDR2) comprising X₁X₂X₃X₄X₅X₆X₇X₈X₉X₁₀, wherein X₁ comprises L or R, X₂ comprises A, G, I, L, M, W, F, P, or V, X₂ comprises A, G, I, L, M, W, F, P, or V, X₄ comprises C, S, T, Y, N, or Q, X₅ comprises C, S, T, Y, N, or Q, X₆ comprises D, E, A, G, I, L, M, W, F, P, or V, X₇ comprises C, S, T, Y, N, or Q, X₈ comprises C, S, T, Y, N, Q, A, G, I, L, M, W, F, P, or V, X₉ comprises D, E, C, S, T, Y, N, or Q, X₁₀ comprises A, G, I, L, M, W, F, P, Y or V; and
(f) a light chain complementarity determining region 3 (LCDR3) comprising X₁X₂X₃X₄X₅X₆X₇X₈X₉, wherein X₁ comprises C, S, T, Y, N, or Q, X₂ comprises C, S, T, Y, N, or Q, X₃ comprises A, G, I, L, M, W, F, P, or V, X₄ comprises A, H, I, K, L, M, P, R, S, V, or Y, X₅ comprises C, F, I, L, R, S, T, Y, N, or Q, X₆ comprises A, G, I, L, M, W, F, P, or V, X₇ comprises A, G, I, L, M, W, F, P, or V, X₈ comprises C, S, T, F, Y, N, or Q, and X₉ comprises C, S, T, Y, N, or Q.

2. The antigen-binding protein of claim 1, wherein:
(a) HCDR1 comprises GFTFSNYA (SEQ ID NO:60) or differs at no more than one or two positions; and/or
(b) HCDR2 comprise IGSGGHYT (SEQ ID NO:61) or differs at no more than one or two positions; and/or
(c) HCDR3 comprises TRETDGYX₈X₉X₁₀X₁₁X₁₂ (SEQ ID NO:81), wherein X₈ comprises D or G, X₉ comprises G or P, X₁₀ comprises I, L, or M, X₁₁ comprises D or L, and X₁₂ comprises L, V, or Y; and/or
(d) LCDR1 comprises QSISDY (SEQ ID NO:62) or differs at no more than one or two positions; and/or
(e) LCDR2 comprises X₁X₂X₃X₄X₅X₆SX₈X₉X₁₀, wherein X₅ comprises A, F, G, H, P, Q, S, T, V, or Y, and X₆ comprises A, D, E, G, L, M, P, S, V, or Y; and/or
(f) LCDR3 comprises QX₂GX₄X₅FPX₈T (SEQ ID NO:82), wherein X₂ comprises S, N, or Q, X₄ comprises A, H, I, L, M, P, S, V, or Y, X₅ comprises F, I, L, Q, R, S, or V, X₈ comprises F or Y.

3. The antigen binding protein of claim 1, wherein in LCDR2, X₁ comprises L or R, X₂ comprises I, L, or V, X₃ comprises I or F, X₄ comprises F, K, N, or Y, X₈ comprises A, F, G, H, P, Q, S, T, V, or Y, X₆ comprises A, D, E, G, L, M, P, S, V, or Y, X₈ comprises A, D, G, L, P, Q, S, V, or Y, X₉ comprises E, G, K, L, Q, R, S, T, of Y, and X₁₀ comprises A, D, I, L, K, N, Q, R, S, or V.

4. The antigen binding protein of claim 1, which comprises no more than two (2) substitutions per CDR as compared to an HCDR1, HCDR2, and HCDR3 depicted in FIG. 3 and an LCDR1, LCDR2, and LCDR3 depicted in FIG. 4.

5. The antigen binding protein of claim 1, which comprises no more than one (1) substitution per CDR as compared to an HCDR1, HCDR2, and HCDR3 depicted in FIG. 3 and an LCDR1, LCDR2, and LCDR3 depicted in FIG. 4.

6. The antigen binding protein of claim 1, which comprises one or more HCDRs of any one of SEQ ID NO:3, SEQ ID NO:5, SEQ ID NO:8, SEQ ID NO:11, SEQ ID NO:19, SEQ ID NO:20, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56 or SEQ ID NO:58 and one or more LCDRs of SEQ ID NO:4, SEQ ID NO:6, SEQ ID NO:7, SEQ ID NO:9, SEQ ID NO:10, SEQ ID NO:12, SEQ ID NO:13, SEQ ID NO:14, SEQ ID NO:15, SEQ ID NO:16, SEQ ID NO:17, SEQ ID NO:18, SEQ ID NO:21, SEQ ID NO:22, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, or SEQ ID NO:59.

7. The antigen binding protein of claim 1, which comprises the HCDRs of any one of SEQ ID NO:3, SEQ ID NO:5, SEQ ID NO:8, SEQ ID NO:11, SEQ ID NO:19, SEQ ID NO:20, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56 or SEQ ID NO:58 and the LCDRs of SEQ ID NO:4, SEQ ID NO:6, SEQ ID NO:7, SEQ ID NO:9, SEQ ID NO:10, SEQ ID NO:12, SEQ ID NO:13, SEQ ID NO:14, SEQ ID NO:15, SEQ ID NO:16, SEQ ID NO:17, SEQ ID NO:18, SEQ ID NO:21, SEQ ID NO:22, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO 41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, or SEQ ID NO:59.

8. The antigen binding protein of claim 1, which comprises the HCDRs of SEQ ID NO:20 and LCDRs of SEQ ID NO:23, the HCDRs of SEQ ID NO:19 and LCDRs of SEQ ID NO:21, the HCDRs of SEQ ID NO:19 and LCDRs of SEQ ID NO:22, the HCDRs of SEQ ID NO:19 and LCDRs of SEQ ID NO:23, the HCDRs of SEQ ID NO:20 and LCDRs of SEQ ID NO:21, or the HCDRs of SEQ ID NO:20 and LCDRs of SEQ ID NO:22.

9. The antigen binding protein of claim 1, which comprises the HCDRs of SEQ ID NO:24 and LCDRs of SEQ ID NO:25, the HCDRs of SEQ ID NO:26 and LCDRs of SEQ ID NO:27, the HCDRs of SEQ ID NO:28 and LCDRs of SEQ ID NO:29, the HCDRs of SEQ ID NO:30 and LCDRs of SEQ ID NO:31, the HCDRs of SEQ ID NO:32 and LCDRs of SEQ ID NO:33, the HCDRs of SEQ ID NO:34 and LCDRs of SEQ ID NO:35, the HCDRs of SEQ ID NO:36 and LCDRs of SEQ ID NO:37, the HCDRs of SEQ ID NO:38 and LCDRs of SEQ ID NO:39, the HCDRs of SEQ ID NO:40 and LCDRs of SEQ ID NO:41, the HCDRs of SEQ ID NO:42 and LCDRs of SEQ ID NO:43, the HCDRs of SEQ ID NO:44 and LCDRs of SEQ ID NO:45, the HCDRs of SEQ ID NO:46 and LCDRs of SEQ ID NO:47, the HCDRs of SEQ ID NO:48 and LCDRs of SEQ ID NO:49, the HCDRs of SEQ ID NO:50 and LCDRs of SEQ ID NO:51, the HCDRs of SEQ ID NO:52 and LCDRs of SEQ ID NO:53, the HCDRs of SEQ ID NO:54 and LCDRs of SEQ ID NO:55, the HCDRs of SEQ ID NO:56 and LCDRs of SEQ ID NO:57, or the HCDRs of SEQ ID NO:58 and LCDRs of SEQ ID NO:59.

10. The antigen binding protein of claim 1, which comprises the HCDRs of SEQ ID NO:8 and the LCDRs of SEQ ID NO:7, the HCDRs of SEQ ID NO:11 and the LCDRs of SEQ ID NO:7, the HCDRs of SEQ ID NO:5 and the LCDRs of SEQ ID NO:9, the HCDRs of SEQ ID NO:5 and the LCDRs of SEQ ID NO:10, the HCDRs of SEQ ID NO:5 and the LCDRs of SEQ ID NO:12, the HCDRs of SEQ ID NO:5 and the LCDRs of SEQ ID NO:13, the HCDRs of SEQ ID NO:5 and the LCDRs of SEQ ID NO:14, the HCDRs of SEQ ID NO:5 and the LCDRs of SEQ ID NO:15, the HCDRs of SEQ ID NO:5 and the LCDRs of SEQ ID NO:16, the HCDRs of SEQ ID NO:5 and the LCDRs of SEQ ID NO:17, or the HCDRs of SEQ ID NO:5 and the LCDRs of SEQ ID NO:18.

11. The antigen binding protein of claim 1, which comprises a heavy chain framework (FR1H+FR2H+FR3H+FR4H) at least 75%, or at least 80%, or at least 85%, or at least 90%, or at least 93%, or at least 95% identical, or identical to SEQ ID NO:3, SEQ ID NO:5, SEQ ID NO:8, SEQ ID NO:11, SEQ ID NO:19, SEQ ID NO:20, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO:42, SEQ ID NO:44, SEQ ID NO:46, SEQ ID NO:48, SEQ ID NO:50, SEQ ID NO:52, SEQ ID NO:54, SEQ ID NO:56 or SEQ ID NO:58.

12. The antigen binding protein of claim 1, which comprises a light chain framework (FR1L+FR2L+FR3L+FR4L) at least 75%, or at least 80%, or at least 85%, or at least 90%, or at least 93%, or at least 95% identical, or identical to SEQ ID NO:4, SEQ ID NO:6, SEQ ID NO:7, SEQ ID NO:9, SEQ ID NO:10, SEQ ID NO:12, SEQ ID NO:13, SEQ ID NO:14, SEQ ID NO:15, SEQ ID NO:16, SEQ ID NO:17, SEQ ID NO:18, SEQ ID NO:21, SEQ ID NO:22, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, or SEQ ID NO:59.

13. The antigen binding protein of claim 1, which comprises a VH domain at least 80%, or at least 85%, or at least 90%, or at least 93%, or at least 95% identical, or at least 97% identical, or identical to SEQ ID NO:3, SEQ ID NO:5, SEQ ID NO:8, SEQ ID NO:11, SEQ ID NO:19, SEQ ID NO:20, SEQ ID NO:24, SEQ ID NO:26, SEQ ID NO:28, SEQ ID NO:30, SEQ ID NO:32, SEQ ID NO:34, SEQ ID NO:36, SEQ ID NO:38, SEQ ID NO:40, SEQ ID NO 42, SEQ ID NO:44, SEQ ID NO:46, SEQ ID NO 48, SEQ ID NO:50, SEQ ID NO:52, SEQ ID NO:54, SEQ ID NO:56 or SEQ ID NO:58.

14. The antigen binding protein of claim 1, which comprises a VL domain at least 80%, or at least 85%, or at least 90%, or at least 93%, or at least 95% identical, or at least 97% identical, or identical to SEQ ID NO:4, SEQ ID NO:6, SEQ ID NO:7, SEQ ID NO:9, SEQ ID NO:10, SEQ ID NO:12, SEQ ID NO:13, SEQ ID NO:14, SEQ ID NO:15, SEQ ID NO:16, SEQ ID NO: 17, SEQ ID NO:18, SEQ ID NO:21, SEQ ID NO:22, SEQ ID NO:23, SEQ ID NO:25, SEQ ID NO:27, SEQ ID NO:29, SEQ ID NO:31, SEQ ID NO:33, SEQ ID NO:35, SEQ ID NO:37, SEQ ID NO:39, SEQ ID NO:41, SEQ ID NO:43, SEQ ID NO:45, SEQ ID NO:47, SEQ ID NO:49, SEQ ID NO:51, SEQ ID NO:53, SEQ ID NO:55, SEQ ID NO:57, or SEQ ID NO:59.

15. An isolated nucleic acid sequence encoding an anti-IL-5 antibody or antibody fragment of claim 1.

16. A vector that comprises the nucleic acid of claim 15.

17. A recombinant cell which comprises the nucleic acid of claim 15.

18. A cell that expresses the antigen binding protein of claim 1.

19. A method of producing the antigen binding protein that specifically binds to IL-5, which comprises culturing the cell of claim 18 under conditions that result in production of the antigen binding protein.

20. A pharmaceutical composition comprising a therapeutically effective amount of the anti-IL-5 protein of claim 1.

21. A method of suppressing IL-5 mediated activation of an eosinophil, which comprises culturing the eosinophil in the presence of an anti-IL-5 protein of claim 1.

22. A method of suppressing an eosinophil mediated inflammatory response in a subject which comprises administering to the subject a therapeutically effective amount of an anti-IL-5 protein of claim 1.

23. A method of inhibiting binding of IL-5 to IL-5 receptor in a subject, which comprises administering to the subject a therapeutically effective amount of the anti-IL-5 protein of claim 1.

24. The method of claim 22, wherein the subject comprises a feline.

25. A method of detecting IL-5 in a sample comprising incubating the sample with an anti-IL-5 protein of claim 1 and detecting the anti IL-5 protein bound to IL-5 in the sample.
